(19) United States
(12) Patent Application Publication
(10) Pub. No.: US 2005/0249799 A1
(43) Pub. Date: Nov. 10, 2005

(54) Polymeric drug delivery system for hydrophobic drugs

(75) Inventors: Jules S. Jacob, Taunton, MA (US); Michael Bassett, Rumford, RI (US); Marcus A. Schestopol, Providence, RI (US); Edith Mathlowitz, Brookline, MA (US); Avinash Nangia, Wrentham, MA (US); Bennett Carter, Cumberland, RI (US); Peyman Moslemy, Providence, RI (US); Ze'ev Shaked, San Antonio, TX (US); David Enscore, Alpharetta, GA (US); Courtney Sikes, Providence, RI (US)

Correspondence Address: PATREA L. PABST;PABST PATENT GROUP LLP, 400 COLONY SQUARE, SUITE 1200, ATLANTA, GA, 30361, US

(73) Assignee: Spherics, Inc.

(21) Appl. No.: 11/072,098

(22) Filed: Mar. 3, 2005

Related U.S. Application Data
(60) Provisional application No. 60/549,777, filed on Mar. 3, 2004.
(60) Provisional application No. 60/605,201, filed on Aug. 27, 2004.
(60) Provisional application No. 60/650,375, filed on Feb. 4, 2005.

(51) Int. Cl. A61K 9/48; A61K 9/20; A61K 9/14

(52) U.S. Cl. 424/451; 424/464; 424/486; 424/488

(57) ABSTRACT

An oral delivery system for Class II drugs that have low oral bioavailability due to their insolubility in water and slow dissolution kinetics and method for making such a drug delivery system are disclosed herein. The formulation may be a controlled release or immediate release formulation. The immediate release formulation contains a Class II drug, together with a hydrophobic polymer, preferably a bioadhesive polymer. In one embodiment, the drug and polymer are co-dissolved in a common solvent. The solution is formed into small solid particles by any convenient method, particularly by spray drying. The resulting particles contain drug dispersed as small particles in a polymeric matrix. The particles are stable against aggregation, and can be put into capsules or tableted for administration. The controlled release formulations contain a BCS Class II drug and a bioadhesive polymer. The controlled release formulations may be in the form of a tablet, capsules, mini-tab, microparticulate, or osmotic pump. Enhancement of oral uptake of the drug from use of bioadhesive polymers occurs through (1) increased dissolution kinetics due to stable micronization of the drug, (2) rapid release of the drug from the polymer in the GI tract; and (3) prolonged GI transit due to bioadhesive properties of the polymers. The combination of these effects allows the preparation of a compact, stable dosage form suitable for oral administration of many class II drugs.

### CROSS-REFERENCE TO RELATED APPLICATIONS

This application claims priority to U.S. Ser. No. 60/549,777, entitled “Polymeric Drug Delivery System for Hydrophobic Drugs”, filed Mar. 3, 2004; U.S. Ser. No. 60/605,201, entitled “Mucoadhesive Oral Formulations of High Permeability, Low Solubility Drugs”, filed Aug. 27, 2004; and U.S. Ser. No. 60/650,375, entitled “Mucoadhesive Oral Formulations of High Permeability, Low Solubility Drugs”, filed Feb. 4, 2005.

### FIELD OF THE INVENTION

The present application is directed to the field of drug delivery, more specifically to the delivery of hydrophobic drugs.

### BACKGROUND OF THE INVENTION

The Biopharmaceutical Classification System (BCS), originally developed by G. Amidon, separates pharmaceuticals for oral administration into four classes depending on their solubility and their absorbability. “Class II” drugs of the BCS system dissolve poorly in the gastrointestinal (GI) tract, but are readily absorbed from solution. Such drugs tend to show a significant difference in their eventual absorption, depending on whether the patient is recently fed versus fasting when taking an oral dose. These drugs may also pass through the GI tract with variable proportions of absorption. These effects make oral formulations of Class II drugs both important and difficult.

Three of the parameters that can be manipulated to improve the bioavailability of Class II drugs are (1) particle size, (2) particle dispersion, and (3) release rate. A variety of methods are available for providing drugs in a form which has a large surface, especially as small particles of a few microns in diameter or smaller. Besides fine grinding of crystals, the formation of microparticles from solution by precipitation, spray drying, freeze-drying, and similar methods is known. In addition, the drug solution can be coated onto small particles to achieve its dispersion, as described, for example, in U.S. Pat. No. 5,633,015 to Gilis et al.

Micronized drug on its own tends to re-agglomerate when administered, and this decreases the advantage of improved release kinetics obtained by micronization. Hence, it is also necessary to prevent fine particles of drug from aggregating in formulation. Polymers and other excipients may form a matrix that separates the micronized particles as they are released. Generally, hydrophilic materials, whether polymers or small molecules, are mixed with the fine particles either during or after manufacture. The dried composite materials are typically tableted or put in a capsule. Then, when the capsule or tablet enters the stomach or intestine, the finely dispersed drug is dispersed into the gastrointestinal fluid without aggregating. Such compositions are sometimes referred to as “immediate release”.

Immediate release solid oral dosage forms are typically prepared by blending drug particles with fillers, such as lactose and microcrystalline cellulose; glidants, such as talc and silicon dioxide; disintegrants, such as starch, crosprovidone; and/or lubricants, such as magnesium stearate; and compressing the mixture into the form of a tablet. Alternately the mixture may be filled into a standard capsule, providing a simple oral dosage form.

Hydrophilic polymers may also be used to form a matrix with hydrophobic drugs to separate drug particles, improve wetting and improve dissolution. Polymers such as hydroxylpropylcellulose (HPC), hydroxpropylmethylcellulose (HPMC), and carboxymethylcellulose (CMC) are commonly used for this purpose. The matrix may be formed by blending and direct compression, hot melt extrusion, spray-drying, spray-congealing, wet granulation and extrusion-spheronization.

Although these techniques are effective in the abstract, the rate of absorption is dependant on whether or not the patient ate when taking the drug. For example, the absorption of the drug is significantly higher when the drug is taken with a meal than when it is not. This may be due to competition between dissolution of drug, and aggregation of drug particles as the water-soluble material dissolves. The latter effect may be minimized in the presence of food.

U.S. Pat. No. 6,509,038 to Baert et al., which proposes another technique for administration, notes these defects in the prior art. This patent advocates resolving these problems by melting the drug and a hydrophilic polymer together, at temperatures of up to 300° C., and then extruding the melted composition. However, ratios of 5 parts of polymer per part of drug are needed, which makes it difficult to make tablets or capsules that can be swallowed by a patient.

Other known biologically-compatible hydrophobic polymers, such as polyglycolic-lactic acid (PLGA) or polylactic acid (PLA), can encapsulate micronized drugs. While these materials typically do not dissolve in water, they do form a coating that retards the rate of release from the matrix system. Such materials are often used to provide controlled-release formulations. However, many Class II drugs absorb or dissolve so slowly that the formulation may pass beyond the absorbing regions of the intestine before be released. Moreover, a system containing a coating formed of a hydrophobic polymer may be especially sensitive to the rates of stomach and intestinal clearance, and thus affected by the timing of meals and other factors as well.

Some controlled release formulations for BCS Class II drugs are available. For example, an extended release tablet for nifedipine is manufactured by Pfizer (PROCARDIA XL®). However, the bioavailability of these drugs and the variability of the formulations can be improved.

Therefore it is an object of the invention to provide drug formulations for oral administration with improved adsorption in the GI tract.

It is a further object of the invention to provide a method for making oral drug formulations with improved adsorption in the GI tract.

### BRIEF SUMMARY OF THE INVENTION

An oral delivery system for Class II drugs that have low oral bioavailability due to their insolubility in water and slow dissolution kinetics and method for making such a drug delivery system are disclosed herein. The formulation may be a controlled release or immediate release formulation. The immediate release formulation contains a Class II drug, together with a hydrophobic polymer, preferably a bioadhesive polymer. In one embodiment, the drug and polymer are co-dissolved in a common solvent. The solution is formed into small solid particles by any convenient method, particularly by spray drying. The resulting particles contain drug dispersed as small particles in a polymeric matrix. The particles are stable against aggregation, and can be put into capsules or tableted for administration. The controlled release formulations contain a BCS Class II drug and a bioadhesive polymer. The controlled release formulations may be in the form of a tablet, capsules, mini-tab, microparticulate, or osmotic pump. Enhancement of oral uptake of the drug from use of bioadhesive polymers occurs through (1) increased dissolution kinetics due to stable micronization of the drug, (2) rapid release of the drug from the polymer in the GI tract; and (3) prolonged GI transit due to bioadhesive properties of the polymers. The combination of these effects allows the preparation of a compact, stable dosage form suitable for oral administration of many class II drugs.

### BRIEF DESCRIPTION OF THE DRAWINGS

FIG. 1 is a cross-section of a trilayer tablet containing BCS II drugs in a central matrix of hydrophilic, rate controlling polymers. The inner core is surrounded on two sides by mucoadhesive polymer layers, optionally surrounded by an enteric coating.

FIG. 2 is a cross section of a longitudinally compressed tablet containing BCS Class II drugs and excipients, and optionally dissolution enhancers, composed in a single monolithic layer that is coated peripherally with a mucoadhesive polymer.

FIG. 3 is a cross-section of a longitudinally compressed tablet containing BCS Class II drugs and excipients, and optionally dissolution enhancers, composed in a single monolithic layer or multiple monolithic layers that is coated peripherally with a mucoadhesive polymer.

FIG. 4 is a cross-section of a longitudinally compressed tablet containing BCS Class II drugs and excipients, and optionally dissolution enhancers, composed in two or three monolithic layers, which are separated by one ore more plugs. The tablet is optionally coated entirely with a moisture-protective polymer then sealed peripherally with a layer of mucoadhesive polymer.

FIG. 5 is a cross-section of a longitudinally compressed tablet that functions as an osmotic delivery system. The BCS Class II drugs and excipients, optionally including dissolution enhancers, are composed in a single core matrix.

FIG. 6 is a cross-section of a longitudinally compressed tablet that functions as push-pull, osmotic delivery system. The core contains one layer of drug and another layer of swelling polymer to push drug out of the tablet at controlled rates.

FIG. 7 is a cross-section of a longitudinally compressed tablet containing precompressed inserts of drug, excipients, and optionally permeation enhancers, embedded in a matrix of mucoadhesive polymer.

FIG. 8 is a cross section of a longitudinally compressed tablet containing BCS Class II drugs and excipients, and optionally dissolution enhancers, composed in a single matrix in which one or more cylindrical pre-compressed reservoirs of drugs are embedded. The tablet is coated peripherally with a mucoadhesive polymer.

FIG. 9 is a cross section of a longitudinally compressed tablet containing BCS Class II drugs and excipients, and optionally dissolution enhancers, composed in two or three monolithic layers, which are separated by one or more fast-dissolving passive matrices. The tablet is coated peripherally with a mucoadhesive polymer to seal the drug layers while the passive matrix is left unsealed.

FIG. 10 is a cross section of a trilayer tablet containing BCS Class II drugs in a single layer or multiple layers of hydrophilic rate controlling polymers. The tablet is coated entirely with one inner layer of a hydrophobic polymer and one outer layer of a mucoadhesive polymer.

FIG. 11 is a graph which shows release rates of itraconazole from a formulation as a function of time, at various levels of loading of the formula with itraconazole.

FIG. 12 is a graph which compares serum levels of itraconazole at two drug loading levels, in the fed and the fasted state.

FIG. 13 is a graph of time (minutes) versus average % itraconazole released for 250 mg tablets (n=6) containing 60% w/w of 33.3% (w/w) Itraconazole/p(AA)/HPMC E5 top sprayed on MCC, 19.7% w/w MCC, 20.0% w/w AcDiSol, and 0.3% w/w Magnesium Stearate in a USP II dissolution bath at a paddle speed of 100 RPM.

FIG. 14 is a graph of the average % intraconazole released from tablets (♦) and gelatin capsules (▪) over time (minutes) when placed in a USP II dissolution bath (n=3) at a paddle speed of 100 RPM. The tablets contained 60.0% w/w of 33.3% (w/w) Itraconazole/p(AA)/HPMC E5 top sprayed on MCC, 19.7% w/w Spray Dried Lactose, 20.0% w/w AcDiSol, and 0.3% w/w Magnesium Stearate; and each capsule contained two tablets.

FIG. 15 is a graph of the average % intraconazole released from gelatin capsules over time (minutes) when placed in a USP II dissolution bath (n=3) at a paddle speed of 100 RPM. The gelatin capsules contained a granulation containing 33.3% w/w Itraconazole/p(AA)/HPMC E5 top sprayed on MCC, 21.7% w/w Polyadipic Acid, 11.7% w/w HPMC E5, and33.3% w/w MCC Cellphere.

FIG. 16 is a graph of the average % intraconazole released from HPMC capsules over time (minutes) when placed in a USP II dissolution bath (n=3) at a paddle speed of 100 RPM. The HPMC capsules contained a granulation containing 33.3% w/w Itraconazole/p(AA)/HPMC E5 top sprayed on MCC, 21.7% w/w Polyadipic Acid, 11.7% w/w HPMC E5, 33.3% w/w MCC Cellphere.

FIG. 17 is a graph of time (hours) versus mean itraconazole plasma concentration following a single dose of Treatment A (Spherazole™ IR) or a single dose of Treatment C (Sporanox® 100 mg Capsule, Janssen, USA).

FIG. 18 is a graph of time (hours) versus mean intraconazole plasma concentration (ng/mL) for Sporanox® (▪for fed state, ● for fasted state) and Spherazole™ IR (□ for fed state, o for fasted state) (n=6), administered to dogs in the in the fed and the fasted state.

FIG. 19 is a graph of time (hours) versus versus mean intraconazole plasma concentration (ng/mL) for Spherazole™ IR (♦) and Spherazole™ CR(▪) (n=6), administered to dogs in the in the fed state.

FIGS. 20A and 20B are graphs of time (hours) versus versus mean intraconazole plasma concentration (ng/mL) for Spherazole™ CR Lot 406-069 dosed to fed beagle dogs.

FIGS. 21A and 21B are graphs of time (hours) versus versus mean intraconazole plasma concentration (ng/mL) for Spherazole™ CR Lot 406-087 dosed to fed beagle dogs.

FIG. 22 is a graph of time (hours) versus versus mean intraconazole plasma concentration (ng/mL) for Spherazole™ CR Lot 406-089 dosed to fed beagle dogs.

FIG. 23 is a graph of time (hours) versus versus mean intraconazole plasma concentration (ng/mL) for Spherazole™ CR Lot 407-007 dosed to fed beagle dogs.

FIG. 24 is a graph of time (hours) versus versus mean intraconazole plasma concentration (ng/mL) for Spherazole™ CR Lot 404-109 dosed to fed beagle dogs.

FIG. 25 is a graph of time (hours) versus versus mean intraconazole plasma concentration (ng/mL) for Spherazole™ CR Lot 403-062 dosed to fed beagle dogs.

FIG. 26 is a graph of time (hours) versus versus mean intraconazole plasma concentration (ng/mL) for Spherazole™ CR Lot 404-096 dosed to fed beagle dogs.

FIG. 27 is a graph of time (hours) versus versus mean intraconazole plasma concentration (ng/mL) for Spherazole™ CR Lot 404-108 dosed to fed beagle dogs.

FIGS. 28A and 28B are box plots is a graph showing a comparison of AUC's (FIG. 28A) and Cmax values (FIG. 28B) of four Spherazole™ CR formulations vs. Sporonox®.

FIGS. 29A, 29B, and 29C are graphs showing a comparison of acyclovir plasma concentrations (μg/mL) over time (hours)and the corresponding AUC, Cmax, and Tmax values of BioVir™ II and Zovirax® (FIG. 29A), BioVir™ and a non-adhesive control (FIG. 29B), and BioVir™, BioVir™+IR formulation, and Zovirax® (FIG. 29C).

### DETAILED DESCRIPTION OF THE INVENTION

Oral delivery compositions for drugs that have low oral bioavailability due to their insolubility in water and slow dissolution kinetics (e.g. Class II drugs) and methods for making and using these compositions are described herein.

I. Compositions

The composition contains a drug with low aqueous solubility and a hydrophobic polymer, preferably a bioadhesive polymer. Optionally the drug is encapsulated in or dispersed throughout a microparticle or nanoparticle. Excipients will typically be included in the dosage form. A wide range of known excpients may be included in the composition.

In one embodiment, the composition is an immediate release formulation. As used herein “immediate release” or “IR” refers to a formulation that releases at least 85% (wt/wt) of the drug within 60 minutes in vitro (under the conditions used in the BCS classification system).

In a second embodiment, the composition is a “controlled release” formulation. As used herein “controlled release” or “CR” refers to a formulation that releases drug more slowly than an IR formulation, i.e. it takes greater than 60 minutes to release at least 85% (wt/wt) of the drug in vitro (under the conditions used in the BCS classification system).

A. Drugs

According to the BCS, drug substances are classified as follows:
Class I—High Permeability, High Solubility
Class II—High Permeability, Low Solubility
Class III—Low Permeability, High Solubility
Class IV—Low Permeability, Low Solubility

The interest in this classification system stems largely from its application in early drug development and then in the management of product change through its life-cycle. In the early stages of drug development, knowledge of the class of a particular drug is an important factor influencing the decision to continue or stop its development.

The solubility class boundary is based on the highest dose strength of an immediate release (“IR”) formulation and a pH-solubility profile of the test drug in aqueous media with a pH range of 1 to 7.5. Solubility can be measured by the shake-flask or titration method or analysis by a validated stability-indicating assay. A drug substance is considered highly soluble when the highest dose strength is soluble in 250 ml or less of aqueous media over the pH range of 1-7.5. The volume estimate of 250 ml is derived from typical bioequivalence (BE) study protocols that prescribe administration of a drug product to fasting human volunteers with a glass (about 8 ounces) of water. In the absence of evidence suggesting instability in the gastrointestinal tract, a drug is considered highly soluble when 90% or more of an administered dose, based on a mass determination or in comparison to an intravenous reference dose, is dissolved.

Class II drugs are particularly insoluble, or slow to dissolve, but readily are absorbed from solution by the lining of the stomach and/or the intestine. Prolonged exposure to the lining of the GI tract is required to achieve absorption. Such drugs are found in many therapeutic classes. A class of particular interest is antifungal agents, such as itraconazole.

Based on the BCS, low-solubility compounds are compounds whose highest dose is not soluble in 250 mL or less of aqueous media from pH 1.2 to 7.5 at 37° C. See Cynthia K. Brown, et al., “Acceptable Analytical Practices for Dissolution Testing of Poorly Soluble Compounds”, Pharmaceutical Technology (Dec. 2004).

The permeability class boundary is based, directly, on measurements of the rate of mass transfer across human intestinal membrane, and, indirectly, on the extent of absorption (fraction of dose absorbed, not systemic bioavailability) of a drug substance in humans. The extent of absorption in humans is measured using mass-balance pharmacokinetic studies; absolute bioavailability studies; intestinal permeability methods; in vivo intestinal perfusion studies in humans; and in vivo or in situ intestinal perfusion studies in animals. In vitro permeation experiments can be conducted using excised human or animal intestinal tissue and in vitro permeation experiments can be conducted with epithelial cell monolayers. Alternatively, nonhuman systems capable of predicting the extent of drug absorption in humans can be used (e.g., in vitro epithelial cell culture methods). A drug substance is considered highly permeable when the extent of absorption in humans is determined to be greater than 90% of an administered dose, based on mass-balance or in comparison to an intravenous reference dose. A drug substance is considered to have low permeability when the extent of absorption in humans is determined to be less than 90% of an administered dose, based on mass-balance or in comparison to an intravenous reference dose. An IR drug product is considered rapidly dissolving when no less than 85% of the labeled amount of the drug substance dissolves within 30 minutes, using U.S. Pharmacopeia (USP) Apparatus I at 100 rpm (or Apparatus II at 50 rpm) in a volume of 900 ml or less in each of the following media: (1) 0.1 N HCI or Simulated Gastric Fluid USP without enzymes; (2) a pH 4.5 buffer; and (3) a pH 6.8 buffer or Simulated Intestinal Fluid USP without enzymes.

Many of the known Class II drugs are hydrophobic, and have historically been difficult to administer. Moreover, because of the hydrophobicity, there tends to be a significant variation in absorption depending on whether the patient is fed or fasted at the time of taking the drug. This in turn can affect the peak level of serum concentration, making calculation of dosage and dosing regimens more complex. Many of these drugs are also relatively inexpensive, so that simple formulation methods are required and some inefficiency in yield is acceptable.

In the preferred embodiment the drug is intraconazole or a related drug, such as fluoconazole, terconazole, ketoconazole, and saperconazole. Itraconazole is a Class II medicine used to treat fungal infections and is effective against a broad spetrum of fungi including dermatophytes (tinea infections), candida, malassezia, and chromoblastomycosis. Itraconazole works by destroying the cell wall and critical enzymes of yeast and other fungal infectious agents. Itraconazole can also decrease testosterone levels, which makes it useful in treating prostate cancer and can reduce the production of excessive adrenal corticosteroid hormones, which makes it useful for Cushing's syndrome. Itraconazole is available in capsule and oral solution form. For fingal infections the recommended dosage of oral capsules is 200-400 mg once a day.

Itraconazole has been available in capsule form since 1992, in oral solution form since 1997, and in an intravenous formulation since 1999. Since Itraconazole is a highly lipophilic compound, it achieves high concentrations in fatty tissues and purulent exudates. However, its penetration into aqueous fluids is very limited. Gastric acidity and food heavily influence the absorption of the oral formulation (Bailey, et al., Pharmacotherapy, 10: 146-153 (1990)). The absorption of itraconazole oral capsule is variable and unpredictable, despite having a bioavailability of 55%.

Other suitable drugs include Class II anti-infective drugs, such as griseofulvin and related compounds such as griseoverdin; some anti malaria drugs (e.g. Atovaquone); immune system modulators (e.g cyclosporine); and cardiovascular drugs (e.g. digoxin and spironolactone); and ibuprofen. In addition, sterols or steroids may be used. Drugs such as Danazol, carbamazepine, and acyclovir may also be used in the compositions.

Danazol is derived from ethisterone and is a synthetic steroid. Danazol is designated as 17a-Pregna-2,4-dien-20-yno[2,3-d]-isoxazol-17-ol, has the formula of C₂₂H₂₇NO₂, and a molecular weight of 337.46. Danazol is a synthetic steroid hormone resembling a group of natural hormones (androgens) that are found in the body. Danazol is used in the treatment of endometriosis. It is also useful in the treatement of fibrocystic breast disease and hereditary angioedema. Danazol works to reduce estrogen levels by inhibiting the production of hormones called gonadotrophins by the pituitary gland. Gonadotrophins normally stimulate the production of sex hormones such as estrogen and progestogen, which are responsible for body processes such as menstruation and ovulation.

Danazol is administered orally, has a bioavailability that is not directly dose-related, and a half-life of 4-5 hours. Dosage increases in danazol are not proportional to increases in plasma concentrations. It has been shown that doubling the dose may yield only a 30-40% increase in plasma concentration. Danazol peak concentrations occur within 2 hours, but the therapeutic effect usually does not occur for approximately 6-8 weeks after taking daily doses.

Acyclovir is a synthetic nucleoside analogue that acts as an antiviral agent. Acyclovir is available for oral administration in capsule, tablet, and suspension forms. It is a white, crystalline powder designated as 2-amino-1,9-dihydro-9-[(2-hydroxyethoxy)methyl]-6H-purin-6-one, has an empirical formula of C₈H₁₁N₅O₃ and a molecular weight of 225.

Acyclovir has an absolute bioavailability of 20% at a 200 mg dose given every 4 hours, with a half-life of 2.5 to 3.3 hours. In addition, the bioavailability decreases with increasing doses. Despite its low bioavailability, acyclovir is highly specific in its inhibitory activity of viruses due to its high affinity for thymidine kinase (TK) (encoded by the virus). TK converts acyclovir into a nucleotide analogue which prevents replication of viral DNA by inhibition and/or inactivation of the viral DNA polymerase, and through termination of the growing viral DNA chain.

Carbamazepine is used in the treatment of psychomotor epilepsy, and as an adjunct in the treatment of partial epilepsies. It can also relieve or diminish pain that is associated with trigeminal neuralgia. Carbamazepine given as a monotherapy or in combination with lithium or neuroleptics has also been found useful in the treatment of acute mania and the prophylactic treatment of bipolar disorders.

Carbamazepine is a white to off-white powder, is designated as 5H-dibenz[b,f]azepine-5-carboxamide, and has a molecular weight of 236.77. It is practically insoluble in water and soluble in alcohol and acetone. The absorption of carbamazepine is relatively slow, despite a bioavailability of 89% for the tablet form. When taken in a single oral dose, the carbamazepine tablets and chewable tablets yield peak plasma concentrations of unchanged carbamazepine within 4 to 24 hours. The therapeutic range for the steady-state plasma concentration of carbamazepine generally lies between 4 and 10 mcg/mL.

B. Bioadhesive Polymers

Bioadhesive polymers are included in the formulation to improve gastrointestinal retention via adherence of the formulation to the walls of the GI tract. As used herein “bioadhesion” generally refers to the ability of a material to adhere to a biological surface for an extended period of time. Bioadhesion requires contact between a bioadhesive material and a surface (e.g. tissue and/or cells). Thus the amount of bioadhesive force is affected by both the nature of the bioadhesive material, such as a polymer, and the nature of the surrounding medium. Bioadhesive polymers may be defined as polymers that have an adherence to mucosal tissue of at least about 110 N/m² of contact area (11 mN/cm²). A suitable measurement method is set forth in U.S. Pat. No. 6,235,313 to Mathiowitz et al. Suitable polymers include polylactic acid (2 kDa MW, types SE and HM), polystyrene, poly(bis carboxy phenoxy propane-co-sebacic anhydride) (20:80) (poly (CCP:SA)), alginate (freshly prepared); and poly(fumaric anhydride-co-sebacic anhydride (20:80) (poly (FA:SA)), types A (containing sudan red dye) and B (undyed). Other high-adhesion polymers include p(FA:SA) (50:50) and non-water-soluble polyacrylates and polyacrylamides.

In a preferred embodiment, bioadhesive polymers are typically hydrophobic enough to be non-water-soluble, but contain a sufficient amount of exposed surface carboxyl groups to promote adhesiveness. These include, among others, non-water-soluble polyacrylates and polymethacrylates; polymers of hydroxy acids, such as polylactide and polyglycolide; polyanhydrides; polyorthoesters; blends comprising these polymers; and copolymers comprising the monomers of these polymers. Blending or copolymerization sufficient to provide a certain amount of hydrophilic character can be useful to improve wettability of the materials. For example, about 5% to about 20% of monomers may be hydrophilic monomers. Polyanhydrides are a preferred type of bioadhesive polymer.

Preferably, the polymers are bioerodable, with preferred molecular weights ranging from 1000 to 15,000 kDa, and most preferably 2000 to 5000 Da.

Polyanhydrides are a preferred type of mucoadhesive polymer. The use of certain bioadhesive polymers, particularly polyanhydrides, allows one polymer additive to serve several functions simultaneously to enhance oral uptake. Suitable polyanhydrides include polyadipic anhydride (“p(AA)”), polyfumaric anhydride, polysebacic anhydride, polymaleic anhydride, polymalic anhydride, polyphthalic anhydride, polyisophthalic anhydride, polyaspartic anhydride, polyterephthalic anhydride, polyisophthalic anhydride, poly carboxyphenoxypropane anhydride and copolymers with other polyanhydrides at different mole ratios.

p(AA) is a surface-eroding polymer belonging to the polyanhydride family of bioerodable and biocompatible polymers. The polymer is a low molecular weight (2-8 kDa) thermoplastic polymer that quickly degrades to adipic acid monomer and adipic anhydride (both of which are considered GRAS for food applications) over the course of 24 hrs at physiological pH.

Optionally, the polymer is a blend of hydrophilic polymers and bioadhesive hydrophobic polymers. Sutiable hydrophilic polymers include hydroxypropylmethylcellulose, hydroxypropylcellulose, carboxymethylcellulose, polyvinylalcohols, polyvinylpyrollidones, and polyethylene glycols. The hydrophobic polymer may contain gastrosoluble polymers that dissolve in stomach contents, such as Eudragit E100.

Other mucoadhesive polymers include DOPA-maleic anhydride co polymer, isopthalic anhydride polymer, DOPA-methacrylate polymers, DOPA-cellulosic based polymers, and DOPA-acrylic acid polymers.

Mucoadhesive materials available from Spherics, Inc., Lincoln, R.I., include Spheromer™ I (poly(fumaric acid:sebacic acid) or “FASA”, as described in U.S. Pat. No. 5,955,096 to Mathiowitz et al.), Spheromer™ II (anhydride oligomers, such as Fumaric Anhydride Oligomer and Metal oxides, such as CaO, ferric oxide, magnesium oxide, titanium dioxide, as described in U.S. Pat. No. 5,985,312 to Jacob et al.), and Spheromer™ III (L-DOPA grafted onto butadiene maleic anhydride at 95% substitution efficiency (L-DOPA-BMA)).

Spheromer™ II may be blended with methylmethacrylates, celluloses and substituted celluloses, polyvinylpyrollidones, PEGs, Poly (vinyl alcohols). Alternatively Spheromer II may be blended with other bioadhesive polymers including p(FA:SA), p(AA), and L-DOPA-BMA.

In designing bioadhesive polymeric formulations based on polylactides, polymers that have high concentrations of carboxylic acid are preferred. This can be accomplished by using low molecular weight polymers (Mw 2000), since low molecular weight polymers contain high concentration of carboxylic acids at the end groups.

In addition, polymers that contain a catechol functionality are also bioadhesive. “Catechol” refers to a compound with a molecular formula of C₆H₆O₂ and the following structure: 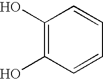
These aromatic groups are substituted for monomers on the backbone of a suitable polymer. The degree of substitution varies based on the desired adhesive strength. It may be as low as 10%, 25%, 50%, or up to 100% substitution. On average, at least 50% of the monomers in a suitable polymeric backbone are substituted with at least one aromatic group. These polymers are available from Spherics, Inc., R.I.

Excipents may also be added to improve bioadhesion. Suitable excipients include FeO/Fe₂O₃, fumaric anhydride pre-polymer (FAPP), L-DOPA-L-DOPA dimer, and adipic anhydride pre-polymer (AAP).

The BCS Class 2 drugs may optionally be encapsulated or molecularly dispersed in polymers to reduce particle size and increase dissolution. The polymers may include polyesters such as poly(lactic acid) or P(LA), polycaprylactone, polylactide-coglycolide or P(LGA), poly hydroxybutyrate poly β-malic acid ); polyanhydrides such as poly(adipic)anhydride or P(AA), poly(fumaric-co-sebacic)anhydride or P(FA:SA), poly(sebacic)anhydride or P(SA); cellulosic polymers such as ethylcellulose, cellulose acetate, cellulose acetate phthalate, etc; acrylate and methacrylate polymers such as Eudragit RS 100, RL 100, E100 PO, L100-55, L100, S100 (distributed by Rohm America) or other polymers commonly used for encapsulation for pharmaceutical purposes and known to those skilled in the art. Also suitable are hydrophobic polymers such as polyimides.

p(AA) prevents coalescence of drug domains within the spray-dried product resulting in increased drug surface area available for dissolution. Additionally, adipic acid monomer generated during polymer degradation increases acidity in the microenvironment of the spray-dried drug particle. By changing the pH, some of the drugs may become more soluble.

Blending or copolymerization sufficient to provide a certain amount of hydrophilic character can be useful to improve wettability of the materials. For example, about 5% to about 20% of monomers may be hydrophilic monomers. Hydrophilic polymers such as hydroxylpropylcellulose (HPC), hydroxpropylmethylcellulose (HPMC), carboxymethylcellulose (CMC) are commonly used for this purpose.

The system can also be designed to extend the time period for release by increasing the drug to polymer ratio, with release drawn out to 80% in 90 minutes (in vitro). Increased relative drug concentration is believed to have the effect of increasing the effective drug domain size within the polymer matrix; and increased drug domain size results in slower drug dissolution. In the case of a polymer matrix containing certain types of hydrophobic polymers, the polymer will act as a mucoadhesive material and increase the retention time of the drug product in the gastrointestinal tract. Increased drug dissolution rates combined with the mucoadhesive properties of the polymer matrix results in (1) increased uptake of the drug and (2) reduction in differences found in the fed and fasted states for the majority of these Class II compounds.

C. Excipients and Additives

The formulation may include one or more excipients. Suitable excipients include solvents, co-solvents, emulsifiers, plasticizers, surfactants, thickeners, pH modifiers, emollients, antioxidants, and chelating agents, wetting agents, and water absorbing agents. The formulation may also include one or more additives, for example, dyes, colored pigments, pearlescent agents, deodorizers, and odor maskers.

Formulations may be prepared using a pharmaceutically acceptable carrier composed of materials that are considered safe and effective and may be administered to an individual without causing undesirable biological side effects or unwanted interactions. “Carrier” as generally used herein refers to all components present in the pharmaceutical formulation other than the active ingredient or ingredients. As generally used herein “carrier” includes, but is not limited to, diluents, binders, lubricants, disintegrants, stabilizers, surfactants, colorants, and fillers.

Diluents, also referred to herein as “fillers”, are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powdered sugar.

Dispersants include, among others water, phosphate-buffered saline (PBS), saline, glucose, sodium lauryl sulfate (SLS), polyvinylpyrrolidone (PVP), polyethylene glycol (PEG), and hydroxypropylmethylcellulose (HPMC).

Binders are used to impart cohesive qualities to a solid dosage formulation, and thus ensure that a tablet, bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose (“HPMC”), microcrystalline cellulose (“MCC”), hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone (PVP).

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (POLYPLASDONE® XL, GAF Chemical Corp.).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-00 monolaurate, polyoxyethylene monolaurate, polysorbates, polyoxyethylene octylphenylether, PEG- 1000 cetyl ether, polyoxycthylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-β-alanine, sodium N-lauryl-β-iminodipropionate, myristoamphoacetate, lauryl betaine and lauryl sulfobetaine.

If desired, the tablets, beads, granules, or particles may also contain minor amount of nontoxic auxiliary substances such as wetting or emulsifying agents, dyes, pH buffering agents, or preservatives.

The BCS Class II drugs may optionally be encapsulated or molecularly dispersed in polymers to reduce particle size. The polymers may include polyesters such as poly(lactic acid), polycaprylactone, poly(lactide-co-glycolide), polyhydroxybutyrate poly(β-malic acid ); polyanhydrides such as poly(adipic)anhydride (“P(AA)”), poly(fumaric-co-sebacic)anhydride (“P(FA:SA)”), poly(sebacic)anhydride (“P(SA)”); cellulosic polymers such as ethylcellulose, cellulose acetate, and cellulose acetate phthalate; acrylate and methacrylate polymers such as EUDRAGIT® RS 100, RL 100, E100 PO, L100-55, L100, S100 (distributed by Rohm America) or other polymers commonly used for encapsulation for pharmaceutical purposes and known to those skilled in the art.

D. Formulations

Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). The formulation may be in the form of a tablet, capsule, minitab, filled tablet, osmotic device, slurry, dispersion, or suspension. In the preferred embodiment, the formulation is a solid oral dosage formulation, such as a tablet, multiparticulate composition, or capsule.

The drug may be incorporated into a polymer matrix at any appropriate loading, such as from 1 to 90% w/w, from 1 to 50 % w/w, from 20 to 70% w/w, from 40 to 60% w/w, from 30 to 40% w/w and preferably in a range from 20% to 30% w/w.

The drug (or pharmaceutically acceptable salts thereof) may be administered in a formulation wherein the drug is in an admixture with one or more pharmaceutically acceptable carriers, excipients or diluents. The pharmaceutical formulations may be produced using standard procedures.

The drug may be complexed with other agents as part of the formulation. The pharmaceutical compositions may take the form of, for example, tablets or capsules prepared by conventional means with pharmaceutically acceptable excipients such as binding agents (e.g., acacia, methylcellulose, sodium carboxymethylcellulose, PVP (Povidone), HPMC, sucrose, starch, and ethylcellulose); fillers (e.g., corn starch, gelatin, lactose, acacia, sucrose, microcrystalline cellulose, kaolin, mannitol, dicalcium phosphate, calcium carbonate, sodium chloride, or alginic acid); lubricants (e.g. magnesium stearates, stearic acid, silicone fluid, talc, waxes, oils, and colloidal silica); and disintegrators (e.g. micro-crystalline cellulose, corn starch, sodium starch glycolate and alginic acid. If water-soluble, such formulated complexes may then be dissolved in an appropriate buffer, for example, phosphate buffered saline or other physiologically compatible solutions. Alternatively, if the resulting complex has poor solubility in aqueous solvents, then it may be formulated with a non-ionic surfactant such as TWEEN™, or polyethylene glycol. Thus, the compounds and their physiologically acceptable solvates may be formulated for administration.

Delayed release and extended release compositions can be prepared. The delayed release/extended release pharmaceutical compositions can be obtained by complexing drug with a pharmaceutically acceptable ion-exchange resin and coating such complexes. The formulations are coated with a substance that will act as a barrier to control the diffusion of the drug from its core complex into the gastrointestinal fluids. Optionaliy, the formulation is coated with a film of a polymer which is insoluble in the acid environment of the stomach, and soluble in the basic environment of lower GI tract in order to obtain a final dosage form that releases less than 10% of the drug dose within the stomach.

Coatings

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Additionally, the coating material may contain conventional carriers such as plasticizers, pigments, colorants, glidants, stabilization agents, pore formers, and surfactants.

Immediate Release Formulations

In one embodiment, the composition is included in an immediate release formulation. Preferably the drug is in the form of nanoparticles or microparticles. The nanoparticles or microparticles are stabilized against aggregation by the hydrophobic polymer; therefore, any of the standard oral dosage forms may be used. A preferred form is encapsulation of the microsphere in a coating that will dissolve in the stomach and/or the intestine.T he nanoparticles or microparticles may be further formulated into tablets, slurries or dispersions for oral administration or placed in capsules, such as gelatin or HPMC capsules.

The BCS Class II drug may be encapsulated in a polymeric matrix. The matrix of polymer is preferably porous, or otherwise allows ready dissolution of the drug in the fluids of the gastrointestinal tract. This allows rapid drug dissolution without reduction in effective particle area by agglomeration of undissolved particles. A matrix that is bioadhesive further enhances absorption by tending to retain the particles in the stomach or upper intestine while the drug is absorbed. The combination of these features allows the uptake of the drug to be relatively independent of the intake of food, or its timing.

Controlled Release Formulations In another embodiment, the composition is included in a controlled release formulation. The controlled release formulations may release at least 80% of the drug in 90 minutes, 4 hours, 12 hours, or up to 24 hours in vitro. The formulation may be designed to release at least 40% of the drug loaded in 30 minutes and at least 70% in 60 minutes in vitro. The controlled release formulations may be designed to release the drug in a pulsatile manner.

The controlled release formulations may be in the form of tablets, capsules, tablets contained in extruded tubing, minitabs, microparticulates, or osmotic pumps. Preferably the tablet is a multilayer tablet, such as a trilayer tablet. In the preferred embodiment, the bioadhesive polymer is a coating on a longitudinally compressed tablet and the BCS Class II drug is in the core of the tablet.

One preferred controlled release formulation contains a BCS Class II granulation that contains at least one binder, such as Eudragit E100 and MCC. The granulation is blended with excipients, such as a rate controlling polymer, a binder, and a lubricant. The granulation is compressed to form a tablet. The preferred bioadhesive layer contains p(FA:SA) (20:80), a rate controlling polymer, and a lubricant. Optionally the bioadhesive layer also contains a pore forming agent.

In the preferred embodiment, the granulation contain 33.3% (w/w) itraconazole, 33.3% (w/w) Eudragit E100, and 33.3% Microcrystalline Cellulose, NF. The granulation is blended with excipients to form a core blend containing 38.9% (w/w) granulation; 15.5% (w/w) Spray-dried lactose, NF; 33.9% (w/w) Methocel Premium LV E5, NF; 11.3% (w/w) Hypromellose 2208 100 cps, NF; and 0.3% magnesium stearate, NF. One preferred bioadhesive layer contains 76.2% (w/w) p(FA:SA) (20:80), 22.8% Eudragit RS PO, NF, and 1% magnesium stearate. A second preferred bioadhesive layer contains 61.3% (w/w) p(FA:SA) (20:80), 22.8% (w/w) Eudragit RS PO, NF, 14.9% (w/w) citric acid anhydrous, USP, and 1% (w/w) magnesium stearate, NF. The preferred tablet contains 42% (w/w) of a bioadhesive layer and 58% (w/w) of the core blend.

II. Methods of Making the Formulations

Solid oral dosage forms are typically prepared by blending powder drug or drug particles (i.e. drug in micro or nanoparticles) with excipients such as those discussed above and compressing the mixture into the form of a tablet. Alternately the mixture may be incorporated into standard pharmaceutical dosage forms such as gelatin capsules and tablets. Gelatin capsules, available in sizes 000, 00, 0, 1, 2, 3, 4, and 5, from manufacturers such as Capsugel®, may be filled with mixtures and administered orally. Similarly, macrospheres may be dry blended or wet-granulated with diluents such as microcrystalline cellulose, lactose, cabosil and binders such as hydroxypropylmethylcellulose, hydroxypropylcellulose, carboxymethylcellulose and directly compressed to form tablets. The dimensions of the tablets are limited only by the engineering of dies available for tabletting machines. Dies to form tablets in round, oblong, convex, flat, and bullet designs in sizes ranging from 1 to 20 mm are available. The resulting tablets may weigh from 1 to 5,000 mg and carry macrospheres at loadings of I to 80% w/w.

The resulting tablets may be coated with sugars, enteric polymers or gelatin to alter dissolution of the tablet. Premature dissolution of the tablet in the mouth may be prevented by coating with hydrophilic polymers, such as hydroxypropylmethylcellulose or gelatin, resulting in dissolution in the stomach.

The tablet or solid oral dosage form may optionally contain absorption enhancers including: sodium caprate, ethylenediamine tetra (acetic acid) (EDTA), citric acid, lauroylcarnitine, palmitoylcarnitine, tartaric acid, Vitamin E TPGS and other agents known to increase GI permeability by affecting integrity of tight junctions.

Drug release rates may be controlled by varying the proportion of drug to carrier in the solution used to prepare the formulation. For example, in some formulations, a drug-polyanhydride system can release drug rapidly, with at least 40% of the drug load in 30 minutes and at least 70% in 60 minutes (in vitro). Drugs are incorporated into the polymer matrix at loadings of 1 to 50% w/w and most preferably in the range of 20-30% w/w.

The composition can also be designed to extend the time period for release by increasing the drug to carrier ratio, with release drawn out to 80% in 90 minutes (in vitro). Increased relative drug concentration is believed to have the effect of increasing the effective drug domain size within a polymer matrix; and increased drug domain size results in slower drug dissolution. In the case of a polymer matrix containing certain types of hydrophobic polymers, the polymer will act as a mucoadhesive material and increase the retention time of the drug in the gastrointestinal tract. Increased drug dissolution rates combined with the mucoadhesive properties of the polymer matrix results in (1) increased uptake of the drug and (2) reduction in differences found in the fed and fasted states for BCS Class II drugs.

A. Formation of Drug Particles

The drug-polymer matrices may be fabricated using any of the encapsulation methods known to those skilled in the art, including but not limited to: solvent evaporation, solvent removal, spray-drying, phase-inversion encapsulation, spontaneous emulsification, coacervation, hot melt encapsulation, hot melt extrusion, spray-congealing, prilling and grinding. It is understood that the drug-polymer products may be further processed into oral dosage form using any of the standard pharmaceutical techniques including but not limited to tabletting, extrusion-spheronization and fluidized bed coating for multiparticulate dosage forms and capsule-filling.

Because the primary source of adhesiveness and of prevention of aggregation is the nature of the polymer(s) forming the microspheres, the exact method of preparation is not critical. The preferred method is spray drying of a solution in which the polymer and the drug are dissolved due to its simplicity. Other suitable methods include spray drying of a solution containing dissolved polymer and dispersed fine particles of drug or freeze-drying of a solution containing dissolved polymer and dissolved or suspended drug. Another method involves dissolving a polymer and dissolving or suspending a drug, and then diluting with a large volume (5× to 20×, for example) of a non-solvent for the polymer and the drug, where the solvent is substantially miscible with the non-solvent (at 20×, at least about 8 to 10% soluble). In preferred pairs of solvents and non-solvents, the absolute values of the differences in solubility parameter “delta” between the solvent and the non-solvent is less than about six. (Delta has units of square root of [calories/cm³]).

In one embodiment, the composition contains a drug/polymer mixture co-dissolved in a mutual solvent and then spray-dried to form microparticles in the range of 2-100 μm in diameter. Drug loadings can range from 0.5-60% (w/w) drug with polymer, but are typically in the range of about 30% to 40%. Polymer systems contain polymers with bioadhesive qualities, and in the preferred embodiment may include either pure polyanhydride polymers, or mixtures of other biocompatible polymers (e.g., methacrylates, polyesters, polysaccharides) with polyanhydrides. The polymer system acts as a matrix for more rapid dissolution of the drug due to increased surface area by maintaining the micronized drug particle size. Spray dried polymer/drug product is then incorporated with suitable pharmaceutical excipients for capsule formation as an oral dose form.

1. Spray Drying

In one embodiment, the composition contains a drug/polymer mixture co-dissolved in a mutual solvent and then spray-dried to form microparticles in the range of 2-100 μm in diameter. Drug loadings can range from 1 to 90% w/w, from 1 to 50% w/w, from 20 to 70% w/w, from 40 to 60% w/w, from 30 to 40% w/w and preferably in a range from 20% to 30% w/w. Polymer systems contain polymers with mucoadhesive qualities, and in the preferred embodiment may include either pure polyanhydride polymers, or mixtures of other biocompatible polymers (e.g., methacrylates, polyesters, polysaccharides) with polyanhydrides. The polymer system acts as a matrix for more rapid dissolution of the drug due to increased surface area by maintaining the micronized drug particle size. Spray dried polymer/drug product is then incorporated with suitable pharmaceutical excipients for capsule formation as an oral dose form.

2. Solvent Evaporation

In this method the polymer is dissolved in a volatile organic solvent, such as methylene chloride. The drug (either soluble or dispersed as fine particles) is added to the solution, and the mixture is suspended in an aqueous solution that contains a surface active agent such as poly(vinyl alcohol). The resulting emulsion is stirred until most of the organic solvent is evaporated, leaving solid particles. Several different polymer concentrations can be used, including concentrations ranging from 0.05 to 0.20 g/ml. The solution is loaded with a drug and suspended in 200 ml of vigorously stirred distilled water containing 1% (w/v) poly(vinyl alcohol) (Sigma). After 4 hours of stirring, the organic solvent evaporates from the polymer, and the resulting particles are washed with water and dried overnight in a lyophilizer. Particles with different sizes (1-1000 microns) and morphologies can be obtained by this method. This method is useful for relatively stable polymers like polyesters and polystyrene.

However, labile polymers, such as polyanhydrides, may degrade during the fabrication process due to the presence of water. For these polymers, the following two methods, which are performed in completely anhydrous organic solvents, are more useful.

3. Hot Melt Microencapsulation

In this method, the polymer is first melted and then mixed with the solid particles of dye or drug that have been sieved to less than 50 microns. The mixture is suspended in a non-miscible solvent (like silicon oil), and, with continuous stirring, heated to 5° C. above the melting point of the polymer. Once the emulsion is stabilized, it is cooled until the polymer particles solidify. The resulting particles are washed by decantation with petroleum ether to give a free-flowing powder. Particles with sizes between one to 1000 microns are obtained with this method. The external surfaces of spheres prepared with this technique are usually smooth and dense. This procedure is used to prepare particles made of polyesters and polyanhydrides. However, this method is limited to polymers with molecular weights between 1000 and 50,000 Da.

4. Solvent Removal

This technique is primarily designed for polyanhydrides. In this method, the drug is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make particles from polymers with high melting points and different molecular weights. Particles that range between 1-300 microns can be obtained by this procedure. The external morphology of spheres produced with this technique is highly dependent on the type of polymer used.

5. Extrusion-Spheronization

Core particles may be prepared by the process of granulation-extrusion-spheronization. In this process, micronized drug is mixed with microcrystalline cellulose, binders, diluents and water and extruded as a wet mass through a screen. The result is rods with diameters equal to the opening of the extrusion screen, typically in the size range of 0.1 to 5 mm. The rods are then cut into segments of approximately equal length with a rotating blade and transferred to a spheronizer. The spheronizer consists of a rapidly rotating, textured plate which propels rod segments against the stationary walls of the apparatus. Over the course of 1-10 minutes of spheronization, the rods are slowly transformed into spherical shapes by abrasion. The resulting spheroid cores are then discharged from the machine and dried at 40-50° C. for 24-48 hours using tray-driers or fluidized bed dryers. The cores may then be coated with rate-releasing, enteric or mucoadhesive polymers using either pan-coating or fluidized-bed coating devices.

B. Preferred Controlled Release Formulations

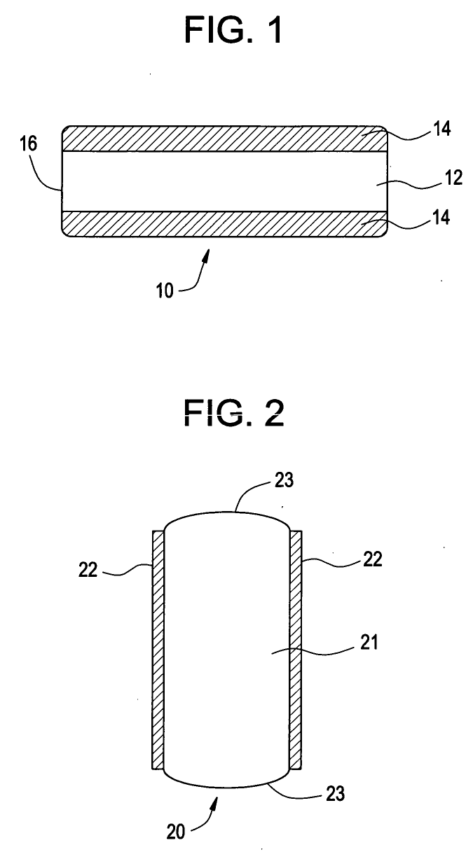

In a preferred embodiment, the solid oral dosage form is a tablet, preferably a trilayer tablet, 10, containing BCS Class II drugs in a central matrix containing excipients, such as fillers or binders, 12 (FIG. 1). The inner core is surrounded on two sides by a mucoadhesive polymer or mixture of mucoadhesive polymers, 14. Optionally, the tablet is coated with an enteric coating, 16.

In another embodiment, the solid oral dosage form is a longitudinally compressed tablet, 20, containing BCS Class II drugs, excipients, and dissolution enhancers, composed in a single monolithic layer, 21. The tablet is sealed peripherally with a layer of mucoadhesive polymer, 22, leaving the upper and lower sides, 23, of the tablet available for drug release. First-order and, more advantageously, zero-order release profiles are achievable with this tablet design. It is feasible to create different release rates for drug by changing the composition of the core matrix. The cross-section of this dosage form is illustrated in FIG. 2.

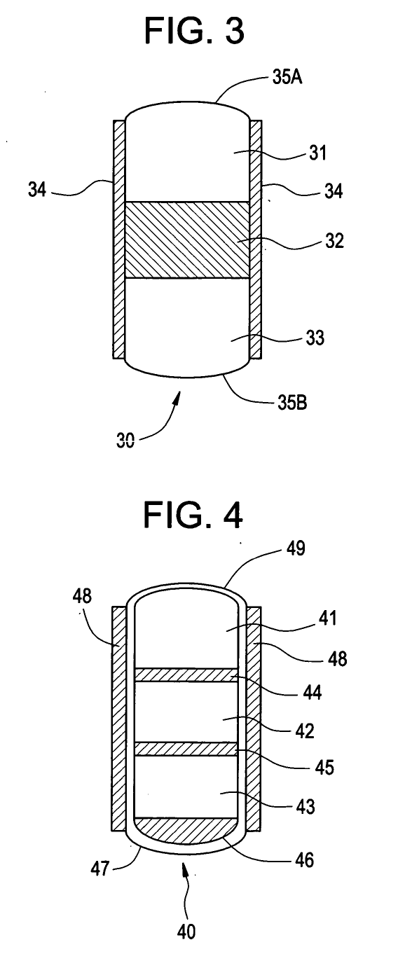

In another embodiment, the solid oral dosage form is a longitudinally compressed tablet, 30, containing BCS Class II drugs, excipients, and dissolution enhancers, composed in a single monolithic layer or multiple monolithic layers, 31-33, which is sealed peripherally with a layer of mucoadhesive polymer, 34, leaving the upper and lower sides, 35A and 35B, of the tablet available for drug release. First-order and, more advantageously, zero-order release profiles are achievable with this tablet design. The tablet can be designed to provide immediate release of the drug and/or extended release rates for the drug by changing the composition of the core matrix or by changing the configuration of their respective layers. The cross-section of this dosage form is illustrated in FIG. 3.

In another embodiment, the solid oral dosage form is a longitudinally compressed tablet, 40, containing BCS Class II drugs, excipients, and dissolution enhancers, composed in two or three monolithic layers, 41-43, which are separated by slow dissolving passive matrices( also referred to herein as “plugs”), 44-46. The tablet is coated entirely with a moisture-protective polymer, 47, and then sealed peripherally with a layer of mucoadhesive polymer, 48, leaving the upper side, 49, of the tablet available for drug release. First-order and, more advantageously, zero-order release profiles are achievable with this tablet design. The tablet can be designed to provide different immediate release or extended release rates for drugs in a two-pulse or three-pulse fashion by changing the composition or configuration of the drug layers, or by changing the formulation or configuration of the plugs. The cross-section of this dosage form is illustrated in FIG. 4.

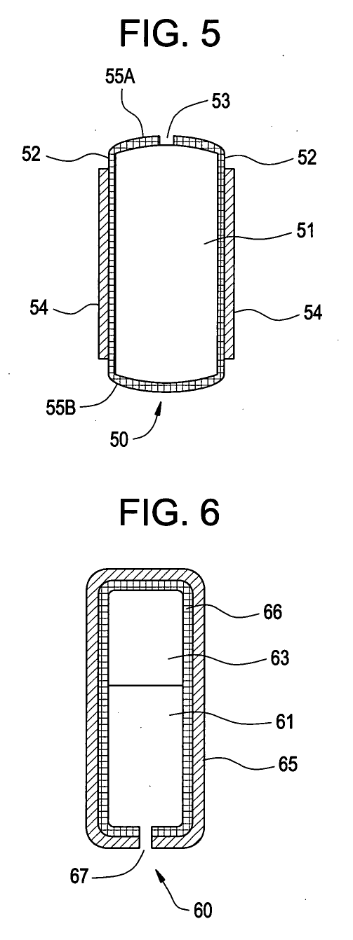

In another embodiment, the BCS Class II drug is delivered from an osmotic delivery system. FIG. 5 illustrates the cross section a longitudinally compressed tablet, 50, based on osmotic controlled delivery containing (1) BCS Class II drugs, excipients, and dissolution enhancers, composed in a single core matrix, 51. The tablet is coated with a semipermeable membrane, 52. One or both sides of the tablet may be perforated, such as by using a micro-drill or a laser beam to make a micrometer-sized orifice, 53. The tablet is sealed peripherally with a matrix of mucoadhesive polymer, 54, leaving the orifice and upper and/or lower sides, 55A and 55B, of the tablet available for drug release. The semipermeable membrane allows permeation of water into the matrix, leading to the dissolution of drug and creation of osmotic pressure. The increase of osmotic pressure will push the drug out of the device through the one or more orifice(s) and membrane at controlled rates. Zero-order release profiles are achievable with this tablet design.

A cross section of an osmotic delivery system of the “push-pull” design is illustrated in FIG. 6. The osmotic delivery system is of the “push-pull” design, 60, and contains a micronized BCS Class II drug and osmotic agents, 61, to draw water across a semi-permeable membrane and a swelling polymer, 63, to push the drug out of the device at controlled rates. The entire device is coated with mucoadhesive polymers, 65, or contains polymer, 66, in the matrix of the capsule. The tablet contains an orifice, 67, through which the drug is delivered.

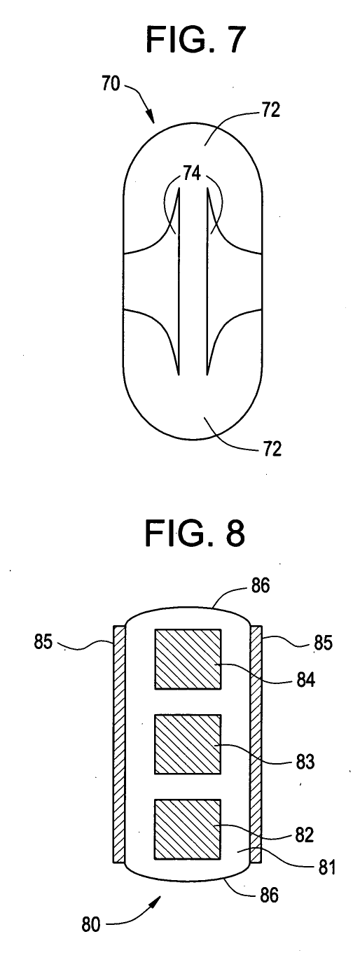

In yet another embodiment illustrated in FIG. 7, a longitudinally compressed tablet, 70, containing precompressed inserts of (1) drug and excipients, 74, and (2) permeation enhancers and excipients, 72, is embedded in a matrix of mucoadhesive polymer. Drug is released only at the edge of the tablet and the kinetics of drug release is controlled by the geometry of the inserts. Zero and first-order release profiles are achievable with this tablet design and it is possible to have different release rates for permeation enhancer and drug by changing the configuration of their respective inserts.

Another embodiment is illustrated in FIG. 8. In FIG. 8, BCS Class II drugs are delivered from a longitudinally compressed tablet, 80, composed in a single matrix, 81, embedding one or more cylindrical pre-compressed inserts, 82-84, consisting of drugs and excipienis, and optionally dissolution enhancers. The tablet is sealed peripherally with a layer of mucoadhesive polymer, 85, leaving the lower and upper sides, 86, of the tablet available for drug release. The tablet can be designed to provide different controlled release or sustained release rates for drugs in a continuous and or pulse mode by changing the formulation or configuration of the inserts.

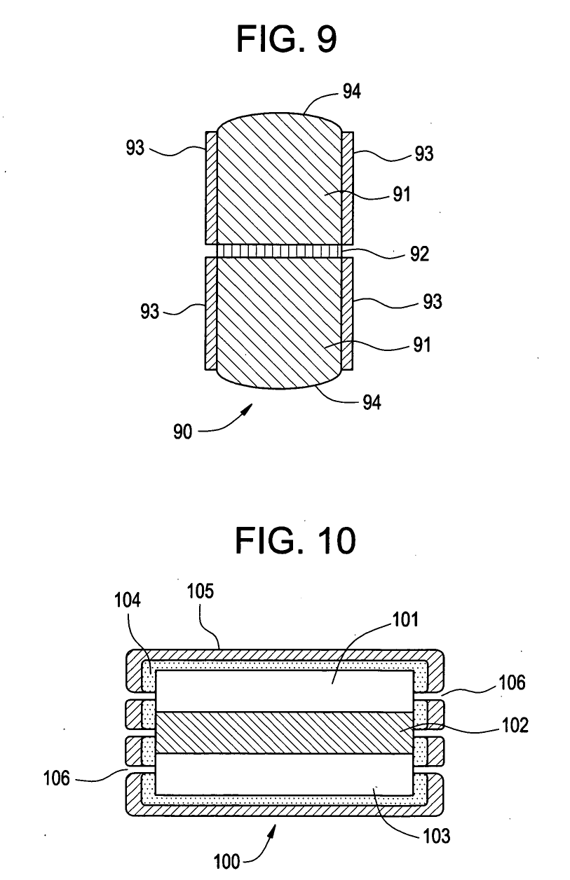

In the embodiment illustrated in FIG. 9, the solid oral dosage form is a longitudinally compressed tablet, 90, containing BCS Class II drugs and excipients, and optionally dissolution enhancers, composed in two or three monolithic layers, 91, which are separated by one or more fast-dissolving passive matrices, 92. The tablet is coated peripherally with a mucoadhesive polymer, 93, sealing the drug layers while leaving the passive matrices unsealed. The upper and lower sides of the tablet, 94, are available for drug release. The tablet is split into two or more segments upon the complete dissolution of the passive matrix, 92, creating new surfaces for dissolution, and thereby, increasing the rate of drug release.

In a further embodiment illustrated in FIG. 10, a conventional tablet, 100, contains one or more layers of BCS Class II drugs and hydrophilic excipients, and optionally dissolution enhancers, 101-103. The tablet is coated entirely first with one layer of a hydrophobic polymer, 104, and second with one layer of a mucoadhesive polymer, 105. Optionally, one or more exit passageways, 106, comprising slits, gashes, notches, or the like, are made on each drug layer along the longer axis of the tablet on one side or on two opposite sides.

III. Uses of BCS Class II Formulations

The oral dosage formulations described herein can be used to treat a variety of diseases and disorders. These formulations have improved bioavailability over formulations that do not contain the bioadhesive polymers. The formulations are designed to facilitate diffusion of drug into intestinal tissue. The formulations can be designed to release drug slowly, quickly or in a step-wise (pulsatile) manner.

The present invention will be further understood by reference to the following non-limiting examples.

### EXAMPLES

### Example 1

### Release of Different Loadings of Itraconazole in Poly(adipic anhydride Coated Compositions Manufactured by Spray Drying

Itraconazole bulk powder and p(AA) were co-dissolved in methylene chloride at varying ratios, to obtain a total solids content of about 8%. The solution was spray dried in a Buchi Spray Dryer Model B-191 using a gas flow rate of 700 lpm, solution flow rate of 10 mL/min, and nozzle temperature at 30° C. Loadings of itraconazole ranged from 10 to 60% (w/w) of the total dry ingredients weight (p[AA] plus Itraconazole), usually in increments of 10%.

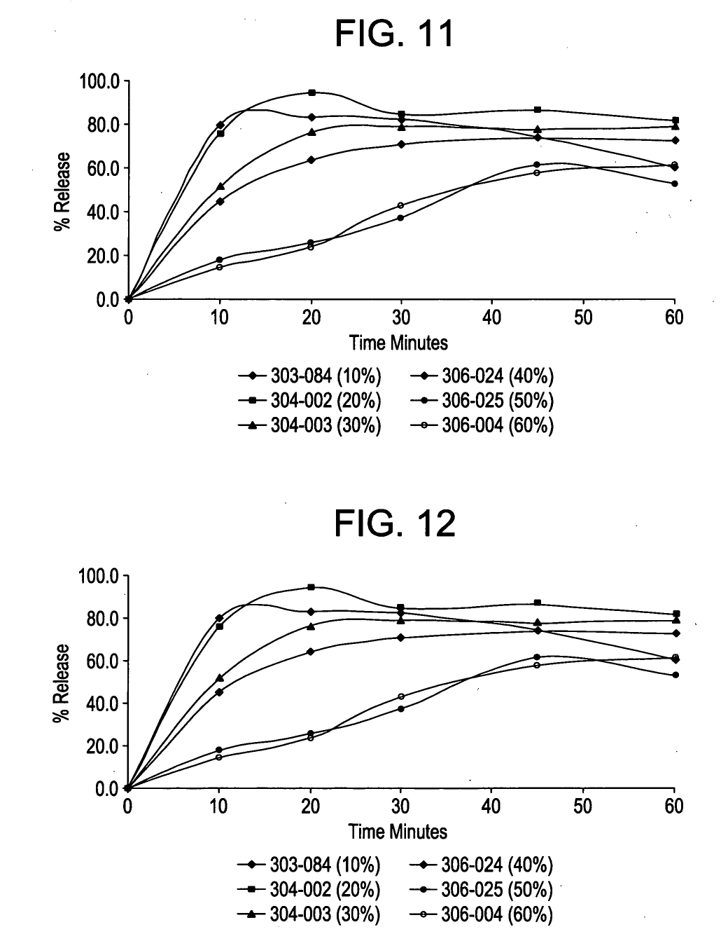

Release rates at 3° C. of intraconazole from the formulations into an aqueous solution buffered at pH 1.2 containing about 1% Tween 80 are shown in FIG. 11. The release rate was found to be slower as the percent loading of the itraconazole increased, particularly above about 40%.

### Example 2

### Plasma Levels of 30% vs. 40% (w/w) Itraconazole/p(AA) Dose Forms in Female Beagle Dogs in the Fed and Fasted States

Four experiments were conducted using retired female breeder beagles that were fed oral dose forms made up of 30 and 40% itraconazole/p[AA] formulations. Dogs were fasted overnight for a minimum of 14 hours; dogs in the “fed” state were given food one-half hour prior to dose administration; “fasted” dogs had food returned 4 hours post-administration. Each cohort contained n=6 dogs. Formulations contained 100 mg of itraconazole; the total amount of itraconazole/p(AA) drug product accounted for 70% (w/w) of the total dose form. The remaining 30% consisted of 1:1:1 of sodium biacarbonate, sodium lauryl sulfate and starch. Doses were packed into 00 gel caps and administered to dogs in the conscious state. 1 mL samples of blood were drawn at 0.5, 1, 2, 4, 6, 8, 24, 48, and 72 hours, placed into pre-heparinized tubes and spun down to collect plasma. Plasma was analyzed for itraconazole content by LC:MS:MS. The results are shown in FIG. 12.

The AUC, Cmax, and Tmax for the results shown in FIG. 12 are listed in Table 1.

TABLE 1

| Formulation/ | AUC | Cmax | Tmax |
|---|---|---|---|
| State | (ng * hr/mL) | (ng/mL) | (hr) |
| 30% fed | 14,830 | 766 | 2 |
| 30% fasted | 12,463 | 383 | 6 |
| 40% fed | 11,404 | 328 | 4 |
| 40% fasted | 5,499 | 140 | 2 |

Results indicate that (1) the fed/fasted differences for a 30% itraconazole/p[AA] formulation are significantly lower than the 2 -3× reported in the literature for the current commercially available form of itraconazole (i.e., Sporanox®, Janssen Pharmaceutica) and (2) the increased release rate of a 30% formulation compared to a 40% formulation correlates directly to the in vivo results observed in dogs.

### Example 3

### Top Spray Drug Layering of Itraconazole/PAA/HPMC E5 onto MCC cores (Lot 407-028)

A granulation containing the composition listed below was prepared using a fluid-bed. The fluid-bed was operated at a set drying temperature of 100° F. at a pump speed of 10 mL/minute and an atomization pressure of 20 psi. The drying air flow at the beginning of the process was set at 50 feet per second (fps) and gradually increased to 72 fps by the end of the process. The outlet temperature varied from 70° F. to 82° F. throughout the experiment.

The granulation contained 33.3% w/w Itraconazole, 21.7% w/w p(AA), 11.7% w/w Methocel Premium LV E5 (HPMC E5), and 33.3% w/w Microcrystalline Cellulose Emocel 90M (MCC).

The resulting granulation was tested for release rate (n=2) in a USP II dissolution bath with a paddle speed of 100 RPM. Granulation samples with a mass of 312 mg were placed in a HCl 0.14N dissolution bath. Approximately 98% of the intraconazole was released within 60 minutes.

### Example 4

### Tablets containing 50 mg of Itraconazole

250 mg tablets containing 60% w/w of 33.3% (w/w) Itraconazole/p(AA)/HPMC E5 top sprayed on MCC (as described in Example 4), 19.7% w/w MCC Avicel® 102 (FMC Corporation), 20.0% w/w AcDiSol, and 0.3% w/w Magnesium Stearate were formed. The tablets were pressed on an Enerpac Minipress with a 0.2618 diameter tablet die and a #91028 tablet punch. The tablets were tested (n=6) for release rate in a USP II dissolution bath at a paddle speed of 100 RPM.

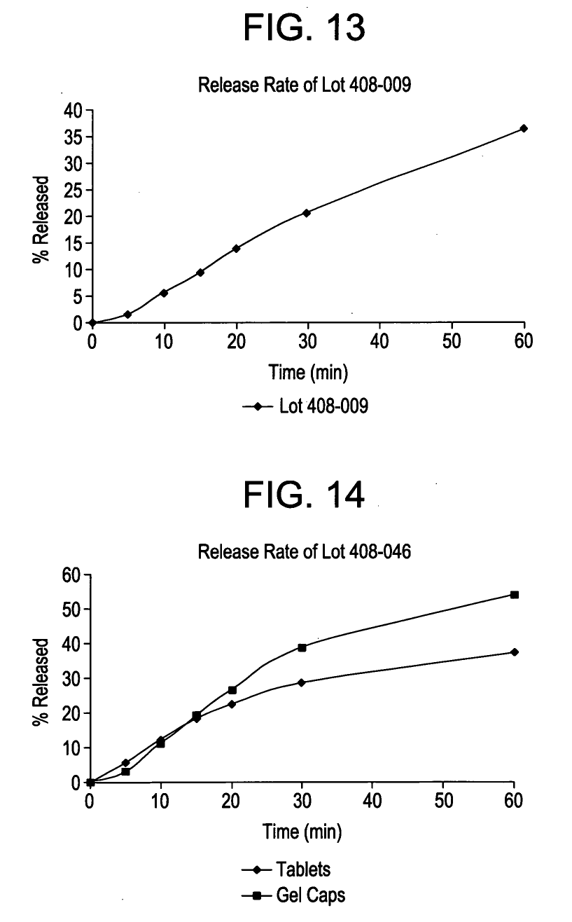

FIG. 13 graphically depicts the average release rate for the tablets over time. The tablets had a nearly linear release profile. After about 1 hour, about 36% of the itraconazole was released.

### Example 5

### Tablets containing 50 mg of Itraconazole (Lot 408-046)

250 mg tablets containing 60.0% w/w of 33.3% (w/w) Itraconazole/p(AA)/HPMC E5 top sprayed on MCC (as described in Example 3), 19.7% w/w Spray Dried Lactose, 20.0% w/w AcDiSol, and 0.3% w/w Magnesium Stearate were formed. The tablets were prepared as described in Example 4. Tablets were also placed in size “0” gelatin capsules. Each capsule contained two tablets.

The tablets and capsules were tested in a USP II dissolution bath (n=3) at a paddle speed of 100 RPM. The dissolution medium contained HCI 0.14N with 0.75% TWEEN® 20.

FIG. 14 graphically depicts the average release rate for the tablets (♦) and gelatin capsules (▪) over time. After about 10 minutes in dissolution medium, the gelatin capsules released itraconazole more quickly than the tablets. After 1 hour, the gelatin capsules had released about 55% of the itraconazole, while the tablets had released about 37%.

### Example 6

### Wurster Coating of MCC with 33% Itraconazole/PAA/HPMC E5/PEG 600 (Lot 409-030)

A granulation was prepared using the Wurster coating method on a fluid bed/granulator. The fluid bed was operated at a set drying temperature of 30° C., and an atomization pressure of 20 psi. The drying air flow was set at fps to begin the process and was gradually increased to 80 fps by the end. The pump speed was 35-45 rpm and the outlet temperature varied from 16.5° C. to 21.3° C. throughout the process.

The granulation contained 33.0% w/w Itraconazole, 19.8% w/w Polyadipic Acid, 11.6% w/w Methocel Premium LV E5, 10.0% w/w Polyethylene Glycol 600, and 25.6% w/w Microcrysalline Cellulose Emocel 90M.

The resulting granulation was tested for release rate (n=6) in a USP II dissolution bath at a paddle speed of 100 RPM. Granulation samples with a mass of 283 mg (100 mg Itraconazole) were placed in a dissolution bath containing HCl 0.14N or HCl 0.14N, with 0.75% TWEEN® 20. The sample placed in HCl 0.14N released about 64 % of the intraconazole after 60 minutes. The samples placed in HCl 0.14N, with 0.75% TWEEN® 20, released only about 32-34% of the intraconazole in the same period of time.

### Example 7

### Gelatin Capsule containing 100 mg Itraconazole (Lot 409-123)

A granulation was prepared using a top spraying fluid bed. The Itraconazole, PAA and HPMC E5 were top-sprayed onto MCC cores. The resulting granulation contained 33.3% w/w Itraconazole, 21.7% w/w Polyadipic Acid, 11.7% w/w HPMC E5, and 33.3% w/w MCC Cellphere. The final granulation was coated with 2.0% w/w Opadry White. The granulation was then placed in a size “0” gelatin capsule.

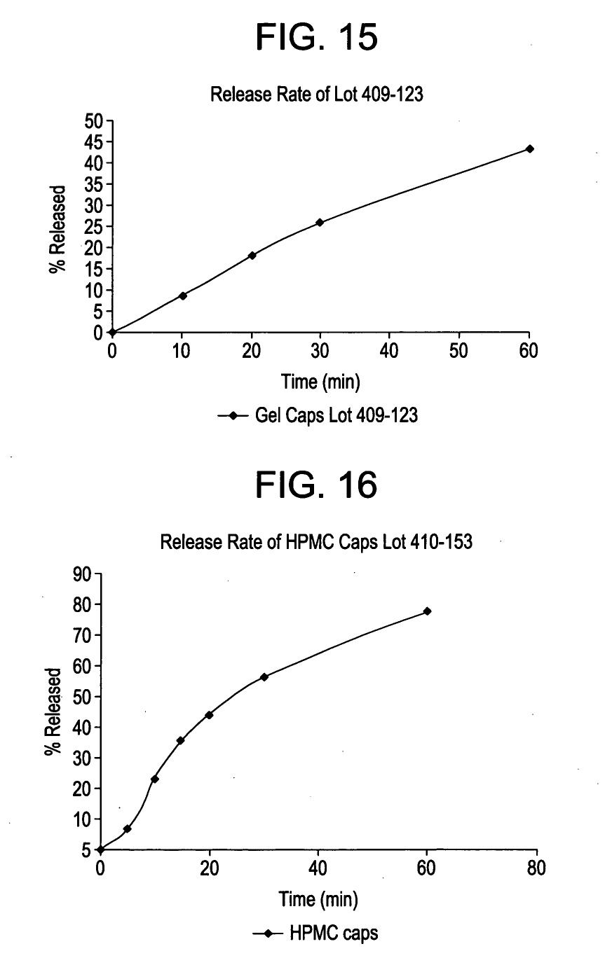

The gelatin capsule (286 mg) was tested for release rate in a USP II dissolution bath containing 0.14N HCl (n=3), at a paddle speed of 100 RPM. FIG. 15 graphically depicts the average release rate for the capsules over time. After 1 hour, the gelatin capsules had released about 45% of the itraconazole.

### Example 8

### HPMC Capsule containing 100 mg Itraconazole. (Lot 410-153)

A granulation was prepared using a top spraying fluid bed as described in Example 9. The granulation was then placed in a size “0” HPMC capsule.

The capsule (286 mg) was tested for release rate in a USP II dissolution bath containing 0.14N HCl (n=3), at a paddle speed of 100 RPM. FIG. 16 graphically depicts the average release rate for the tablets over time. After 1 hour, the capsules had released about 78% of the itraconazole.

### Example 9

### Single-Dosing Bioavailability Testing of Spherazole™ IR Formulation versus Sporanox® in Healthy Human Volunteers

A commercially available intraconazole tablet is marketed by Janssen Pharmaceutica using the trade name Sporanox®. Sporanox® contains of 100 mg of itraconazole coated onto sugar non-pareils, overlayed by a gastrosoluble, hydroxpropylmethylcellulose (HPMC) top coat. Sporanox® is known to have widespread PK and AUC differences between dosings and also demonstrates considerable fed-fasted variability.

A test immediate release formulation (referred to herein as “Spherazole™ IR”) was similar with respect to active pharmaceutical ingredient (API) and dose level. Spherazole™ IR contained 100 mg of itraconazole encapsulated within spray-dried p(AA). The itraconazole/p(AA) complex was then dry-granulated with common tableting excipients such as microcrystalline cellulose (MCC), magnesium stearate, talc, and crocarmellose sodium and then compressed into a tablet using 0.375×0.745 inch modified oval tooling.

TABLE 2
Composition of Spherazole ™ IR

| Component | mg/tablet | % w/w |
|---|---|---|
| Itraconazole, USP | 100 | 11.1% |
| HPMC E5, USP | 99.9 | 11.1% |
| Poly Adipic Anhydride | 133.2 | 14.8% |
| (PAA) | | |
| Microcrystalline | 362.7 | 40.3% |
| Cellulose, NF | | |
| Talc, USP | 17.6 | 2.0% |
| Croscarmellose Sodium, | 177.7 | 19.7% |
| NF | | |
| Magnesium Stearate, NF | 8.9 | 1.0% |
| Total | 900 | 100.0% |

The major difference between the Sporanox® and Spherazole™ IR was the inclusion of p(AA).

For the human studies described below, p(AA)was used as a matrix polymer to micronize drug particles by spray-drying with p(AA). p(AA) prevents coalescence of drug domains within the spray-dried product resulting in increased drug surface area available for dissolution. Additionally, adipic acid monomer generated during polymer degradation increases acidity in the microenvironment of the spray-dried drug particle, which increases dissolution of itraconazole. Dissolution of the drug is negligible above pH 4. The purpose of these formulations was to reduce differences in drug absorption in the fed and fasted digestive states. Another aim of the formulations was to reduce variability between dosings and reduce peak plasma levels (Cmax).

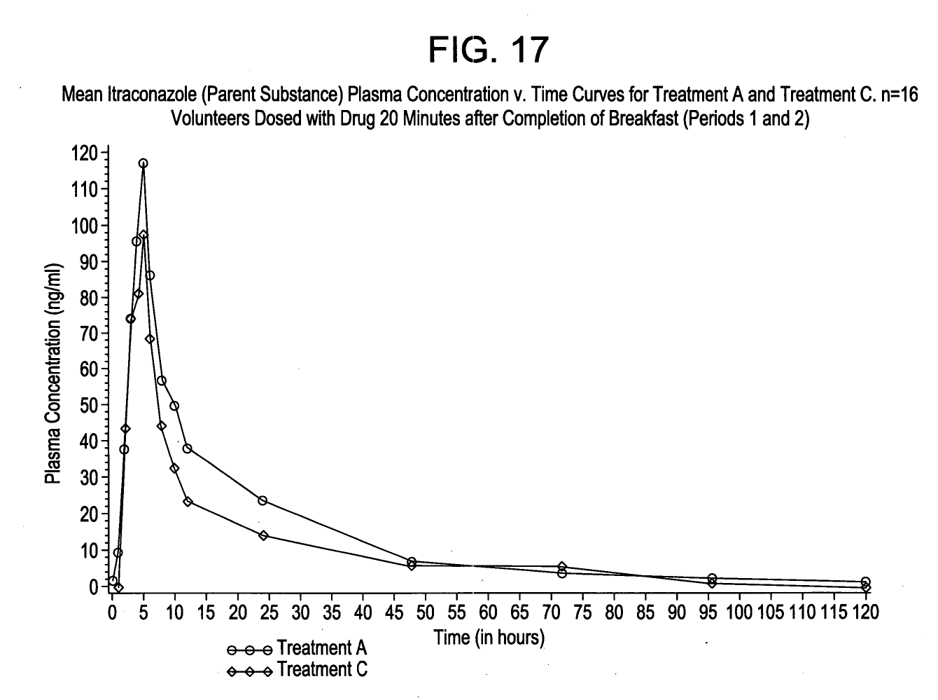

Spherazole™ IR formulation was compared Sporonox® after single dosing in the fed state in 16 volunteers. The tablets were administered to the volunteers 20 minutes after completion of breakfast. The results of the study are graphically depicted in FIG. 17. FIG. 17 is a graph of mean itraconazole plasma concentration versus time following a single dose of Treatment A (Spherazole™ IR) or a single dose of Treatment C (Sporanox® 100 mg Capsule, Janssen, USA).

The results of a statistical analysis of the data obtained in this study are provided in Table 3.

TABLE 3
Statistical Analysis of Intraconazole Plasma data for Spherazole ™ IR (A) and Sporanox ® (C)

| | Arith. Mean ± SD | | Minimum | | Maximum | |
|---|---|---|---|---|---|---|
| Variable | A | C | A | C | A | C |
| Cₘₐₓ (ng/mL) | 128.28 ± 62.10 | 110.93 ± 54.84 | 48.60 | 26.20 | 266.00 | 209.00 |
| AUC₀₋ₜ (ng/mL · h) | 1449.64 ± 646.19 | 1097.28 ± 676.50 | 307.32 | 193.59 | 2765.85 | 2296.11 |
| AUC_{0-∞} (ng/mL · h) | 1818.79 ± 849.16 | 1515.11 ± 952.65 | 403.93 | 224.24 | 3668.88 | 3460.03 |

| | Geometric Mean | | 90% Confidence Limits | Point Estimate |
|---|---|---|---|---|
| Variable | A | C | (CI) | (%) |
| Cₘₐₓ (ng/mL) | 115.09 | 97.16 | 89.66%-156.50% | 118.46% |
| AUC₀₋ₜ (ng/mL · h)* | 1467.94 | 1014.22 | 103.03%-149.96% | 121.28% |
| AUC_{0-∞} (ng/mL · h)* | 1809.10 | 1569.91 | 92.21%-133.39% | 115.15% |

| Variable | Intraindividual CV % | Interindividual CV % |
|---|---|---|
| Cₘₐₓ (ng/mL) | 47.06% | 48.74% |
| AUC₀₋ₜ (ng/mL · h) | NC | 53.00% |
| AUC_{0-∞} (ng/mL · h) | NC | 54.05% |

*Non-parametric,
NC = Not Calculated

Spherazole™ IR had greater bioavailability (AUC=1449.64±646.19 ng/mL*h) than Sporanox® (AUC=1097.28±676.50 ng/mL*h). Examination of the log-transformed data showed significant reductions in variability for the maximum plasma concentration, as indicated by the Cmax value, and bioavailability, as indicated by the Area-under-the-curve when taken out to 120 hours or infinity, for Spherazole™ as compared to Sporonox®.

### Example 10

### Fluoroscopy Study of Barium-Impregnated Trilayer Tablets with Mucoadhesive Polymer Outer Layers

Trilayer tablets were prepared by sequentially filling a 0.3287×0.8937” “00” capsule die (Natoli Engineering) with 333 mg of the following blends: a bioadhesive outer layer blend, followed by inner core blend and finally by bioadhesive outer layer blend. The tablets were compressed at 2000 psi for 1 sec using a Globepharma Manual Tablet Compaction Machine (MTCM-1). The outer layer contained 333 mg of either poly(fumaric acid:sebacic acid 20:80 (p[FA:SA 20:80]) (also referred to herein as “Spheromer I™”) or L-DOPA grafted onto butadiene maleic anhydride at 95% substitution efficiency (L-DOPA-BMA) (also referred to herein as “Spheromer III™”). The inner core contained 233 mg of a blend of hydroxyprbpylmethylcellulose (HPMC) 4000 cps and 100 mg of barium sulfate.

The tablets were administered to female beagles that were fasted for 24 hrs. The tablets were also dosed to fasted beagles that had been fed with chow, 30 minutes before dosing (fed). Tablets were continuously imaged with fluoroscopy over the course of 6 hrs in unrestrained dogs. Trilayer tablets with Spheromer I™ or III™ in the mucoadhesive layers remained in the stomach of fasted dogs for up to 3.5 hrs and resided in the stomach of fed dogs in excess of 6 hrs. The. tablets did not mix with food contents and remained in contact with stomach mucosa at the same location until they passed into the small intestine.

### Example 11

### Comparison of Sporanox®, Spherazole™ IR and Spherazole™ CR Tablets

“Spherazole™ IR” is an immediate release formulation of itraconazole. Itraconazole was spray-dried with poly(fumaric-co-sebacic)anhydride (20:80) (also referred to herein as “Spheromer I”) to reduce drug particle size and blended with excipients including croscarmellose sodium, NF, Talc USP and Magnesium Stearate NF in an 8 qt V shell blender. The blend was dry granulated by slugging, to increase bulk density. The blend was compressed with 0.5906″ round tooling in a Stokes B2 press, to produce slugs with hardness not less than 3 kp. The slugs were sized by forcing the slugs through a #30 mesh sieve. The milled slugs were blended with microcrystalline cellulose, croscarmellose sodium, talc and magnesium stearate. The final blend was compressed with 0.375×0.745″ modified oval tooling using Stokes B2 tooling to produce 900 mg tablets with hardness not less than 8 kp. The final product was a 900 mg oval tablet containing 100 mg of itraconazole, which is the same weight as the Sporanox® dose. The composition of the tablet was 11% (w/w) itraconazole; 14.8% (w/w) poly(adipic anhydride), 11.1% (w/w) HPMC 5 cps (E5), 2% (w/w) Talc, 19.7% (w/w) Cross-linked carboxymethylcellulose sodium (AcDiSOL), 1% (w/w) Magnesium Stearate, and 40.3% (w/w) Microcrystalline cellulose (MCC).

By comparison, Spherazole™ CR is formulated as a trilayer tablet. Itraconazole is dissolved in a dichloromethane with Eudragit E 100 and either spray-dried (SD) or drug-layered onto MCC cores, blended with HPMC of different viscosities (5, 50, 100, or 4000 cps) and other excipients (corn starch, lactose, microcrystalline cellulose or MCC) to control drug release. The rate controlling inner drug layer is then sandwiched between outer adhesive layers composed of Spheromer I or poly(butadiene maleic anhydride) graft L-DOPA (herein referred to as “Spheromer III”) and optionally Eudragit RS PO to improve mechanical properties of the bioadhesive layer. A number of different Spherazole™ CR formulations were tested and are described in more detail in Examples 12-20, below.

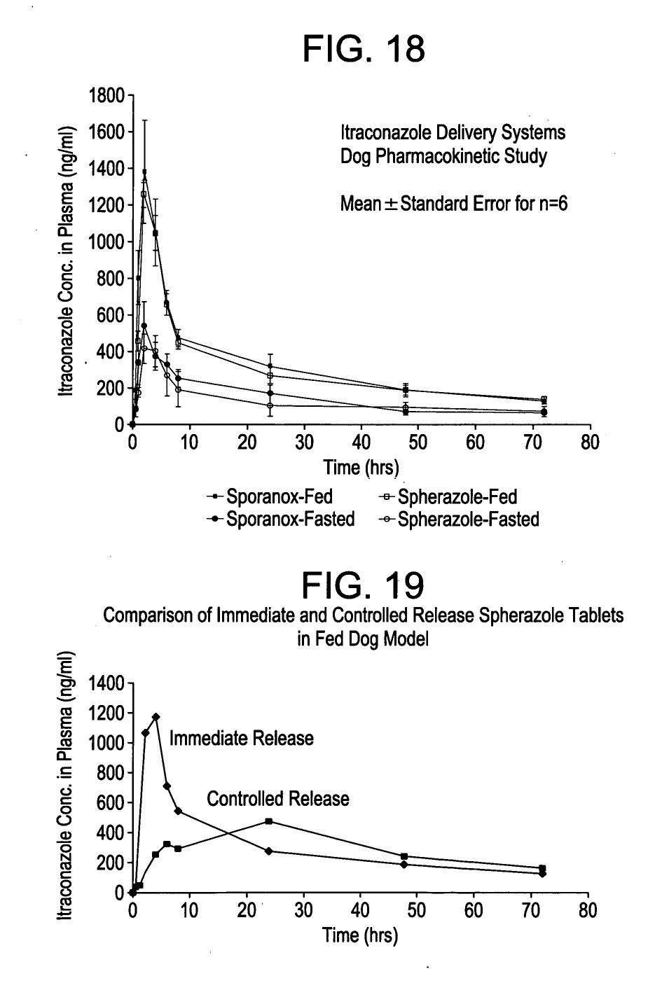

Sporanox®, Spherazole™ IR and Spherazole™ CR were tested in the “fed” beagle model described in Example 10. Sporanox® and Spherazole™ IR were also tested in the “fasted” beagle model described in Example 10. The itraconazole plasma concentrations (ng/mL) at different time points were measured and the mean values were plotted. FIG. 18 provides the PK profiles for Spherazole™ IR (100 mg) and Sporanox® (100 mg). FIG. 19 provides the PK profiles for Spherazole™ IR and a typical Spherazole™ CR formulation (n=6). Area under the plasma concentration versus time curve (AUC), maximum plasma concentration (Cmax) and time to maximum plasma concentration (Tmax) were calculated. Spherazole™ IR has an AUC in the range of 20,000±2000 ng/ml*hr−1, Cmax of 1200±300 ng/ml, Tmax of 2±1 hrs. This performance is equivalent to performance of Sporanox® in the fed dog model and less variable than the innovator product.

The tested Spherazole™ CR formulations have AUC in the range of 20,000±2000 ng/ml*hr−1, Cmax of 600±200 ng/ml, Tmax of 8-20 hrs depending on the particular composition of the rate-controlling core. The performance of Spherazole™ CR formulations is similar to Spherazole™ IR and Sporanox® with respect to AUC. However, Cmax is lower by 50%, which is an important benefit in terms of reduced side effects and drug toxicity. The extended Tmax facilitates once daily dosing (qd dosing) dosing compared to twice dailiy dosing (bid dosing) for Sporanox® and other immediate release products.

### Example 12

### Bioadhesive Trilayer Tablet containing 100 mg Spray-Dried (SD) Itraconazole (Lot 406-069)

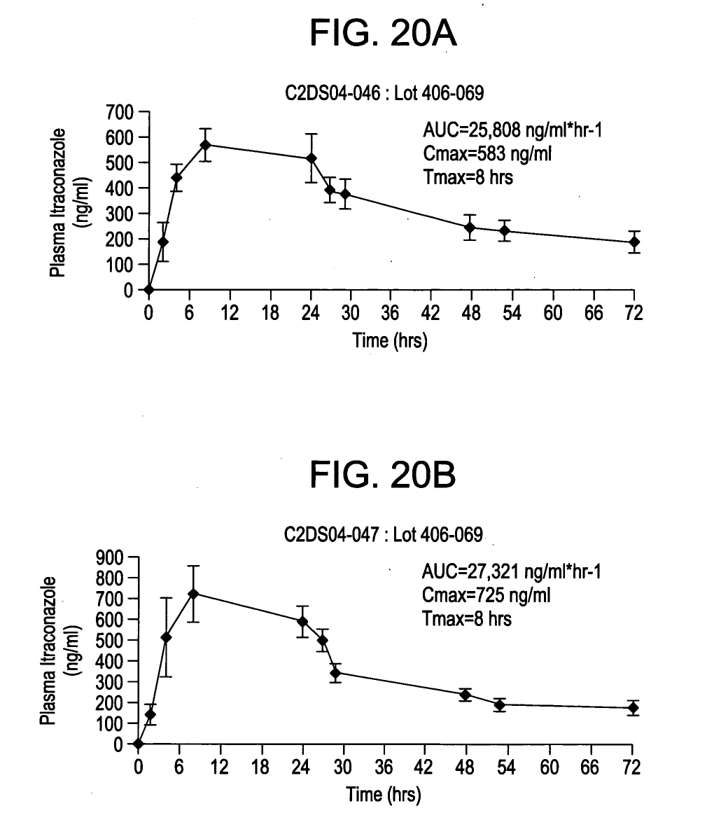

Trilayer tablets were prepared according to the formulation listed below and tested twice (n=6/test) in the fed beagle model. The itraconazole plasma concentrations at different time points were measured and the mean values were plotted on a graph (see FIGS. 20A and 20B). The AUC of this formulation was superior to the AUC range for Spherazole™ IR and Sporanox® in the same model (see Example 11).

Inner Core: (700 mg)

46% w/w 30% Itraconazole/E100 SD
40% w/w HPMC 4000 cps
13.7% w/w Corn Starch 1500
0.7% w/w Magnesium Stearate
   Outer Layer: (200 mg×2)
75% w/w Spheromer I
24% w/w Eudragit RS PO
1% w/w Magnesium Stearate

### Example 13

### Bioadhesive Trilayer Tablet Containing 100 mg Spray-dried Itraconazole (Lot 406-087)

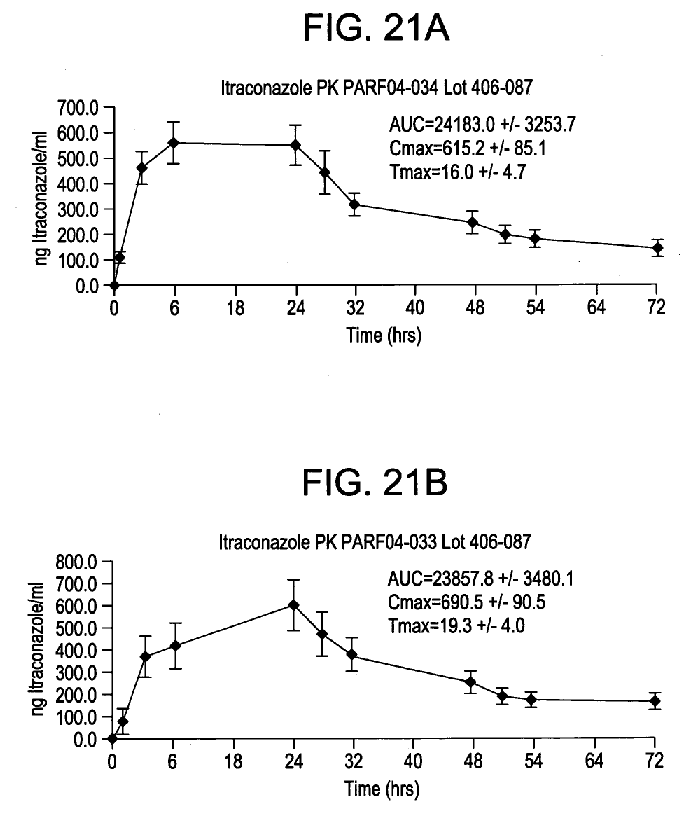

Trilayer tablets were prepared according to a formulation that was the same as the formulation in Example 12, except in the 40% w/w HPMC 4000 cps, in the inner core, was replaced with 20% w/w HPMC 4000 cps and 20% w/w/HPMC 5 cps. The outer core contained the same composition, but the total mass was greater than in Example 3 (250 mg×2). These tablets were tested twice (n=6/test) in the fed beagle model. The itraconazole plasma concentrations at different time points were measured and the mean values were plotted on a graph (see FIGS. 21A and 21B). The AUC of this CR formulation was superior to the AUC range for Spherazole™ IR and Sporanox® in the same model. The AUC and Cmax for this formulation were similar to the AUC and Cmax for Example 12. The Tmax was longer than the Tmax for Example 12.

### Example 14

### Non-Adhesive Trilayer Tablet with 100 mg Spray-Dried Itraconazole (Lot 406-089)

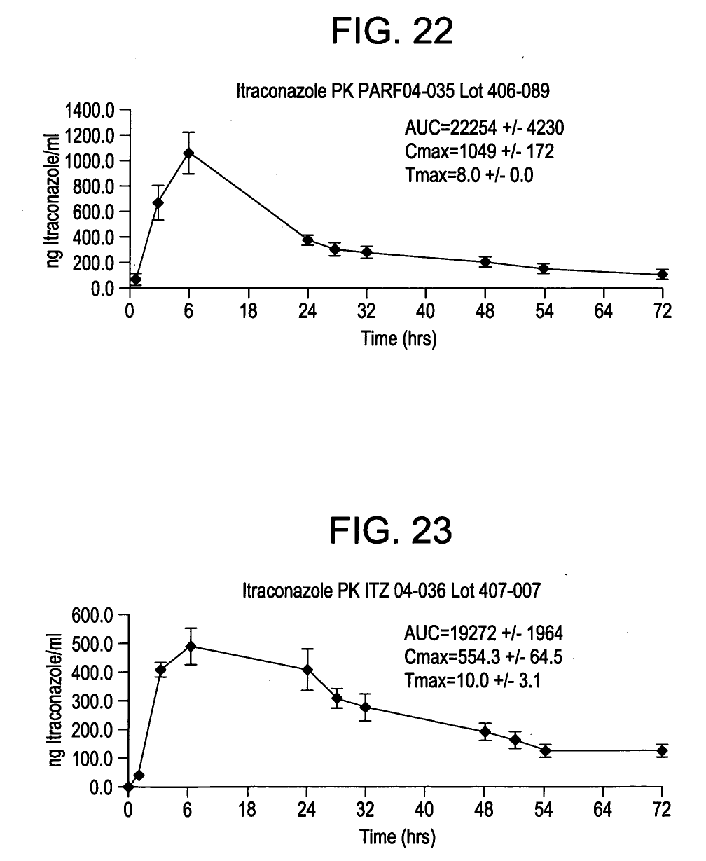

Trilayer tablets were prepared and tested once (n=6/test) in the fed beagle model. This formulation is identical to Lot 406-087 tested in Example 13, except that a non-adhesive polymer, Ethocel 20 cps, was substituted for Spheromer I. The intraconazole plasma concentrations at different time points were measured and the mean values were plotted on a graph (see FIG. 22). The AUC of the non-adhesive formulation was similar to the AUC from adhesive Lot 406-087 (see Example 4), except that Tmax was reduced from 16 and 19 hrs to 8 hrs in the non-adhesive formulation, and the Cmax for the non adhesive formulation was 1049 ng/ml compared to a Cmax of 615 and 691 ng/ml for the adhesive formulation, Lot 406-087 (see Example 13). Using a non-adhesive polymer in the outer layers changed the in vivo performance so that it more closely resembled Spherazole™ IR (see Example 11 and FIG. 19).

### Example 15

### Bioadhesive Trilayer Tablet with 100 mg Spray-Dried Itraconazole

Trilayer tablets were prepared according to the formulation for Example 13, except the itraconazole was layered onto MCC Cores (30% Itraconazole/E100 MCC Cores). These tablets were tested once (n=6/test) in the fed beagle model. The itraconazole plasma concentrations at different time points were measured and the mean values were plotted on a graph (see FIG. 23). AUC of the CR formulation was similar to the AUC range for Spherazole™ IR and Sporanox® in the same model. Cmax was similar to Examples 12 and 13 (Lots 406-069 and 406-087) and Tmax was 10 hrs.

### Example 16

### Bioadhesive Trilayer Tablet with 100 mg Itraconazole Spray-Dried Itraconazole (Lot 404-109)

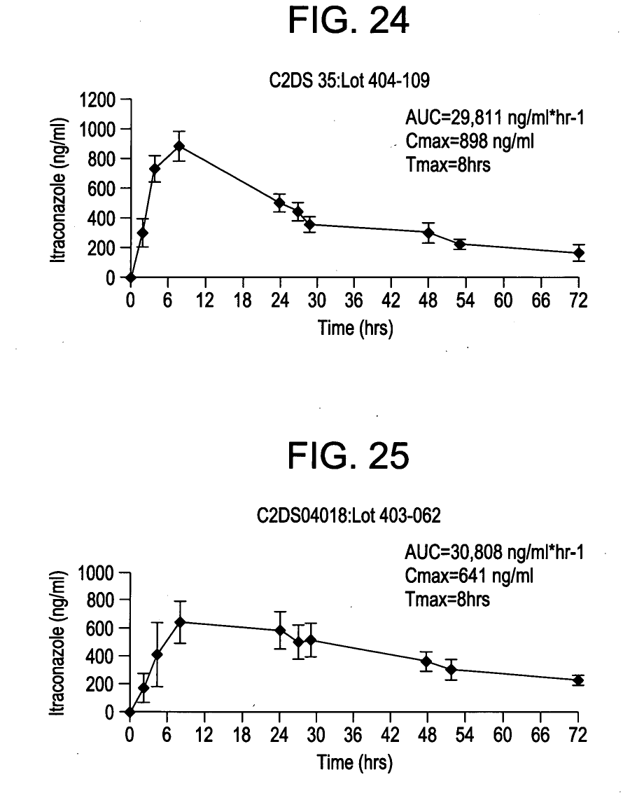

Trilayer tablets were prepared according to the formulation for Example 15, except the ratio of the two HPMC components was modified so that the inner core contained 10% w/w HPMC 4000 cps and 30% w/w HPMC 5 cps. These tablets were tested once (n=6/test) in the fed beagle model. The itraconazole plasma concentrations at different time points were measured and the mean values were plotted on a graph (see FIG. 24). AUC of the CR formulation was similar to the AUC range for Spherazole™ IR and Sporanox® in the same model. Cmax was slightly greater compared to Examples 12 and 13 (Lots 406-069 and 406-087) and Tmax was 8 hrs.

### Example 17

### Bioadhesive Granulation with 100 mg Itraconazole Spray-Dried Itraconazole in Gelatin Capsules (Lot 403-062)

Itraconazole was spray-dried with bioadhesive poly[adipic anhydride co-dissolved in solution dichloromethane to produce 40% Itraconazole w/w loaded particles. The spray drying conditions used were: Inlet temperature 40° C., feed rate 10 ml/min, atomization pressure 40 psi. The spray-dried particles were blended with HPMC 4000 cps and fluid bed granulated using 3% HPMC E5 as the binder. The granulation was filled into “000” gel caps and tested once (n=6/test) in the fed beagle model. The itraconazole plasma concentrations at different time points were measured and the mean values were plotted on a graph (see FIG. 25). AUC of this formulation was superior to the AUC range for Spherazole™ IR and Sporanox® in the same model. Cmax was similar to Examples 12 and 13 (Lots 406-069 and 406-087) and Tmax was 8 hrs.

### Example 18

### Bioadhesive Trilayer Tablet with 100 mg Itraconazole Spray-Dried Itraconazole (Lot 404-096)

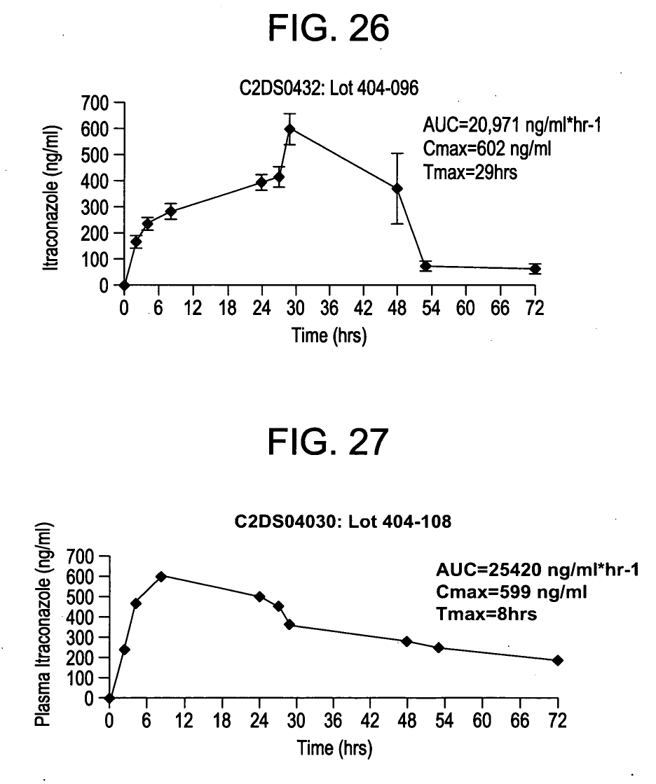

Trilayer tablets were prepared according to the formulation listed below and tested once (n=6/test) in the fed beagle model. The itraconazole plasma concentrations at different time points were measured and the mean values were plotted on a graph (see FIG. 26). AUC of the CR formulation was similar to the AUC range for Spherazole™ IR and Sporanox® in the same model. Cmax was similar to Examples 12 and 13 (Lots 406-069 and 406-087) and Tmax was 29 hrs.

Inner Core: (333 mg)

100% w/w 30% Itraconazole/HPMC E5 spray-dried
   Outer Layer: (333 mg×2)
66% w/w Spheromer III
33% w/w Polyplasdone XL (Crospovidone)
1% w/w Magnesium Stearate

### Example 19

### Bioadhesive Trilayer Tablet with 100 mg Itraconazole Spray-Dried Itraconazole (Lot 404-108)

Trilayer tablets were prepared according to the formulation for Example 16, except the itraconazole was spray dried with Eudragit E100 (30% Itraconazole/E100 SD). The tablets were tested once (n=6/test) in the fed beagle model. The itraconazole plasma concentrations at different time points were measured and the mean values were plotted on a graph (see FIG. 27). AUC of this formulation was similar to the AUC range for Spherazole™ IR and Sporanox® in the same model. Cmax was similar to Examples 12 and 13 (Lots 406-069 and 406-087) and Tmax was 8 hrs.

### Example 20

### Performance of Bioadhesive Trilayer Tablet Formulations with 100 mg Itraconazole Spray-Dried Itraconazole in the Fed Dog Model

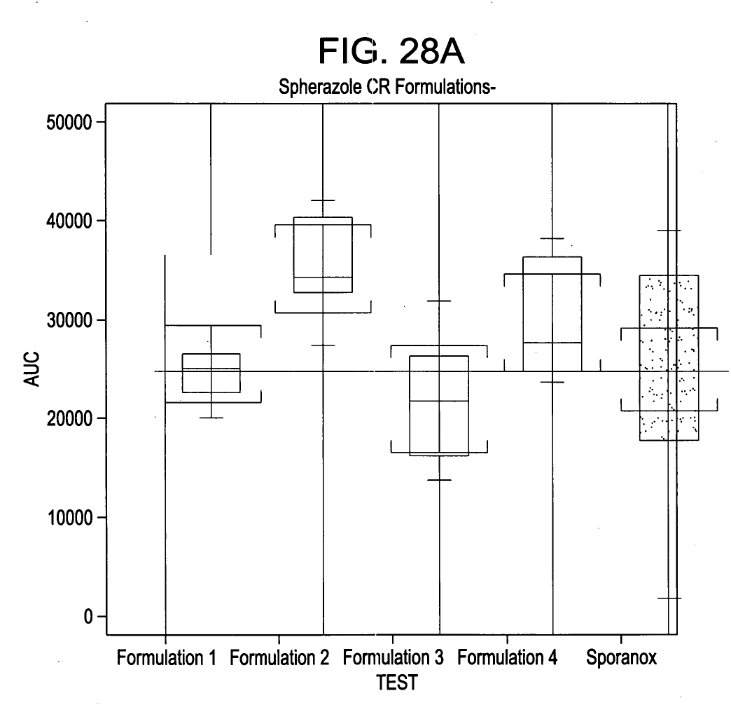

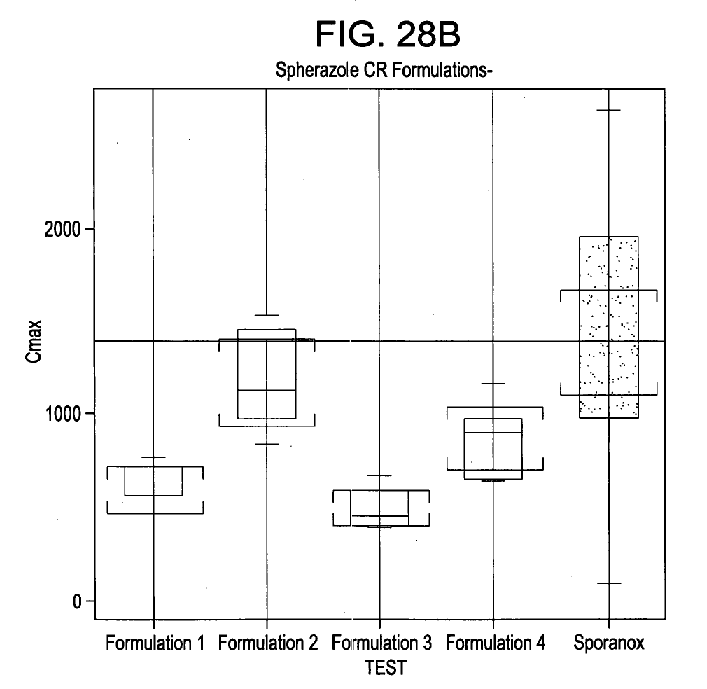

22 Spherazole™ CR formulations, including those described in the Examples listed above, were tested in the fed dog model and four were identified as having considerably lower variability, including Examples 16 and 19, in AUC and Cmax compared to Sporanox®, as depicted in FIGS. 28A and 28B.

FIGS. 28A and 28B are box plots showing the range of individual data points for the AUC (FIG. 28A) and Cmax (FIG. 28B) values obtained for four of the Spherazole™ CR formulations, including Examples 16 and 19, and Sporanox®. The AUC and Cmax values for each of the four formulations had less variability than the AUC and Cmax values for Sporanox®.

### Example 21

### In Vitro Dissolution and PK Performance of Zovirax® 400 mg

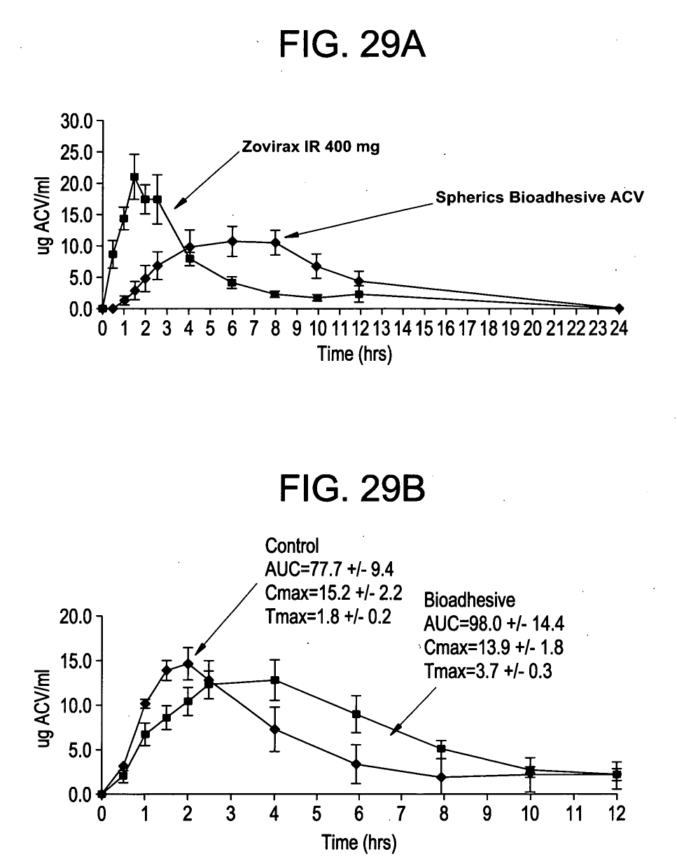

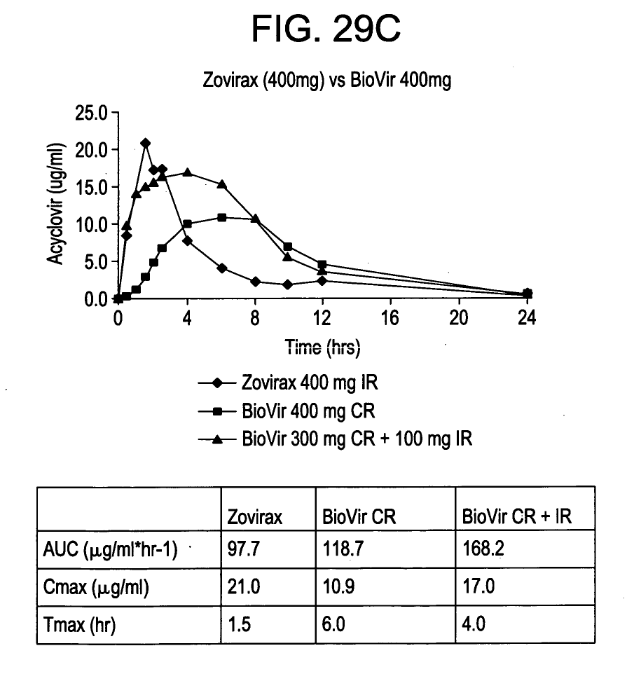

Zovirax® (GlaxoSmithKline) (Acyclovir) 400 mg, Immediate Release (IR) tablet were tested for dissolution in SGF, pH 1.2 in a USP 2 Paddle apparatus at 100 rpm. 100% of the drug was released in 10 minutes. A single 400 mg dose was administered to beagle dogs in the “fed” state and the following PK profile resulted: This data is included in FIG. 29A (▪) and listed in Table 4.

TABLE 4
In Vitro Dissolution of Zovirax ® (400 mg) Tablet

| Time | Mean | | |
|---|---|---|---|
| (hrs) | (% Release) | SD | SE |
| 1 | 0.0 | 0.0 | 0.0 |
| 0.5 | 8.6 | 5.3 | 2.4 |
| 1 | 14.2 | 4.5 | 2.0 |
| 1.5 | 21.0 | 8.0 | 3.6 |
| 2 | 17.4 | 5.2 | 2.3 |
| 2.5 | 17.5 | 8.8 | 3.9 |
| 4 | 7.9 | 2.5 | 1.1 |
| 6 | 4.1 | 1.5 | 0.7 |
| 8 | 2.3 | 0.7 | 0.3 |
| 10 | 2.0 | 1.3 | 0.6 |
| 12 | 2.6 | 2.9 | 1.3 |
| 24 | 0.2 | 0.2 | 0.2 |
| AUC | 97.7 | 30.3 | 13.6 |
| Cmax | 22.6 | 7.7 | 3.4 |
| Tmax | 1.6 | 0.8 | 0.4 |

### Example 22

### In Vitro Dissolution and PK Performance of BioVir™ I (400 mg) )Lot 404-093)

Trilayer tablets (also referred to herein as “BioVir™” I) were prepared using the following formula:

Inner Core: (539 mg)

74% w/w Acyclovir
12.4% w/w HPMC 100 cps
6.2% w/w HPMC 5 cps
3.1% w/w Glutamic Acid (acidulant)
3.1% w/w Corn Starch 1500
0.7% w/w Magnesium Stearate
   Outer Layer: (250 mg×2)
99% w/w Spheromer™ III
1% w/w Magnesium Stearate

BioVir™ (400 mg acylclovir) tablets were tested for dissolution in SGF, pH 1.2 in a USP 2 Paddle apparatus at 100 rpm.

TABLE 5
In Vitro Dissolution of BioVir ™ I (400 mg) Tablet

| Time (min | 404-093 (% Release) |
|---|---|
| 0 | 0 |
| 10 | 5.3 |
| 30 | 12.9 |
| 60 | 29.3 |
| 120 | 55.4 |
| 180 | 75.4 |
| 270 | 90.5 |

A control formulation that was identical to BioVir™ I, except that Spheromer™ III, was replaced with non-adhesive polyethylene, was formed. A single dose of BioVir™ I and of the control were administered to beagle dogs in the “fed” state. This resulting PK profiles for these formulations are provided in FIG. 29B.

TABLE 6
Plasma concentrations of Acyclovir
following administration of BioVir ™ I (400 mg) Tablet

| | Mean | | |
|---|---|---|---|
| Time | (μg/ml) | SD | SE |
| 0 | 0.0 | 0.0 | 0.0 |
| 0.5 | 2.1 | 1.4 | 0.7 |
| 1 | 6.6 | 2.2 | 1.1 |
| 1.5 | 8.5 | 2.6 | 1.3 |
| 2 | 10.4 | 3.2 | 1.6 |
| 2.5 | 12.3 | 3.1 | 1.5 |
| 4 | 12.7 | 4.7 | 2.3 |
| 6 | 9.0 | 3.9 | 2.0 |
| 8 | 5.0 | 1.9 | 1.0 |
| 10 | 2.6 | 1.1 | 0.5 |
| 12 | 2.2 | 1.2 | 0.6 |
| 24 | 0.2 | 0.1 | 0.0 |
| AUC | 98.0 | 28.8 | 14.4 |
| Cmax | 13.9 | 3.6 | 1.8 |
| Tmax | 3.7 | 0.7 | 0.3 |

The AUC of BioVir™ I was identical to Zovirax®, the Cmax was 62% of Zovirax® and the Tmax shifted from 1.6 hrs for Zovirax® to 3.7 hrs for BioVir™ I (see FIGS. 29A and 29B). The AUC of the non-adhesive tablet was lower than Zovirax®, and the Cmax was 69% of Zovirax®.

### Example 23

### In Vitro Dissolution and PK Performance of BioVir™ II 400 mg (Lot 404-093)

Trilayer tablets (also referred to herein as BioVir™ II) were prepared using the following formula:

Inner Core: (600 mg)

67.6% w/w Acyclovir
16.9% w/w Ethocel 10 Standard FP
11.3% w/w Glutamic Acid (acidulant)
2.7% w/w Talc
0.5% w/w Aerosil 200
1.0% w/w Magnesium Stearate
   Outer Layer: (300 mg×2)
99% w/w Spheromer III
1% w/w Magnesium Stearate

BioVir™ II 400 mg, Controlled Release (CR) tablets were tested for dissolution in SGF, pH 1.2 in a USP 2 Paddle apparatus at 100 rpm. This data is depicted listed in Table 7.

TABLE 7
In Vitro Dissolution of BioVir ™ II (400 mg) Tablet

| Time (min) | 404-134 (% Release) |
|---|---|
| 0 | 0 |
| 10 | 3.3 |
| 30 | 7.1 |
| 60 | 11.3 |
| 120 | 20.3 |
| 180 | 27.3 |
| 270 | 37.8 |

A single 400 mg dose was administered to beagle dogs in the “fed” state and the acyclovir plasma concentration s were measured at different time points. The mean plasma concentration for each time point is provided in FIG. 29A.

TABLE 8
Plasma concentrations of Acyclovir
following administration of BioVir ™ II (400 mg) Tablet

| Time | Mean | | |
|---|---|---|---|
| (hrs) | (μg/mL) | SD | SE |
| 0 | 0.0 | 0.0 | 0.0 |
| 0.5 | 0.3 | 0.2 | 0.1 |
| 1 | 1.5 | 0.8 | 0.4 |
| 1.5 | 3.5 | 2.5 | 1.2 |
| 2 | 5.8 | 3.9 | 2.0 |
| 2.5 | 8.2 | 4.3 | 2.1 |
| 4 | 11.8 | 4.1 | 2.0 |
| 6 | 12.1 | 4.6 | 2.3 |
| 8 | 10.4 | 4.8 | 2.4 |
| 10 | 6.0 | 3.9 | 2.0 |
| 12 | 4.1 | 3.5 | 1.8 |
| 24 | 0.2 | 0.2 | 0.1 |

The AUC for BioVir™ II was 118.7±20.1, the Cmax was 13.1±1.8 (mg/mL), and the Tmax was 5.1±1.0 (hrs). The AUC BioVir™ II was higher than for Zovirax®, the Cmax was 59% of the Zovirax® Cmax and the Tmax shifted from 1.6 hrs for Zovirax® to 4.5 hrs for BioVir™ II (see FIG. 29A).

### Example 24

### Comparison of PK Performance for Zovirax®, BioVir™ II, and BioVir™ II±Immediate Release Formulations

A controlled release (CR), trilayer tablet having the composition described above in Example =b 3, and containing 300 mg of acyclovir was produced by direct compression at 3000 psi for 5 seconds. The inner core weighted 444 mg and each outer weighed 225 mg.

An immediate release (IR) tablet containing 100 mg of acyclovir was prepared with the following composition and directly compressed at 2000 psi for 1 second.

IR Tablet Composition:

600 mg
33% Zovirax® granulation
25% Spray-dried lactose
25% Microcrystalline cellulose
16.6% Croscarmellose sodium, NF
0.4% Magnesium Stearate, NF

One tablet of the CR and one tablet of IR formulation were dosed to a fed beagle dog and blood samples were taken different appropriate time intervals.

The PK Profiles for Zovirax® (400 mg acyclovir), BioVir™ II (400 mg acyclovir), and BioVir™ II (300 mg acyclovir)+Immediate Release (100 mg acyclovir) (“IR+CR”)are presented in FIG. 29C. The AUC of the IR+CR dosing was 168.2 μg/ml*hr compared to 97.7 μg/ml*hr for Zovirax®, representing a 72% improvement in AUC. Cmax of the IR+CR dosing was 17.0 μg/ml compared to 21 μg/ml for Zovirax®, and Tmax was 4 hrs compared to 1.5 hrs for Zovirax®.

Those skilled in the art will recognize, or be able to ascertain using no more than routine experimentation, many equivalents to the specific embodiments of the invention described herein. Such equivalents are intended to be encompassed by the following claims.

1. An oral dosage formulation comprising a mixture of a Class II drug and a bioadhesive polymer selected from the group consisting of microparticles of hydrophobic bioadhesive polymer containing drug and controlled release formulations of bioadhesive polymer containing drug.

2. The formulation of claim 1 wherein the drug is selected from the group consisting of anti-fungal drugs, antibiotics, steroids, hormones, and immunosuppressants.

3. The formulation of claim 2 wherein the drug is selected from the group consisting of itraconazole, fluoconazole, terconazole, ketoconazole, saperconazole, griseofulvin, griseoverdin, Danazole, Atovaquone, cyclosporine, digoxin, and spironolactone.

4. The formulation of claim 1 wherein the bioadhesive polymer is water-insoluble and is selected from the group consisting of polyanhydrides, poly(meth)acrylate, polyhydroxy acids, polyesters, and copolymers or mixtures thereof, blends comprising these polymers; and copolymers comprising the monomers of these polymers.

5. The formulation of claim 1 wherein the polymer comprises a mucoadhesive component selected from the group consisting of DOPA-anhydride polymer, DOPA-methacrylate polymers, DOPA-cellulosic based polymers, DOPA-acrylic acid polymers, anhydride oligomers, metal oxides, and DOPA grafted anhydrides.

6. The formulation of claim 1 wherein the formulation releases at least about 40% of the drug from the microparticles into a fluid of the gastrointestinal tract, or into water, in less than about 60 minutes.

7. The formulation of claim 1, wherein the composition is in a form selected from the group consisting of tablets, capsules, minitabs, filled tablets, osmotic devices, slurries, dispersions, and suspensions.

8. The formulation of claim 1 wherein the drug is in the form of particles.

9. The formulation of claim 1 wherein drug is incorporated into polymer at a loading selected from the group consisting of from 1 to 90% w/w, from 1 to 50% w/w, from 20 to 70% w/w, from 40 to 60% w/w, from 30 to 40% w/w and preferably in a range from 20% to 30% w/w.

10. The formulation of claim 1 wherein 80% of the drug is released in 90 minutes in vitro.

11. The formulation of claim 1 further comprising a permeation or asorption enhancer.

12. The formulation of claim 1 wherein the polymer matrix is porous.

13. The formulation of claim 1 wherein the formulation comprises a coating selected from the group consisting of bioadhesive coatings, enteric coatings, sugar coatings, and water-soluble polymer coatings.

14. The formulation of claim 1 comprising a tablet having a drug core and layers of mucoadhesive coating thereon.

15. The formulation of claim 1 comprising a tablet comprising multiple monolithic layers, separated by slow dissolving passive matrices, coated with a moisture-protective polymer, and sealed peripherally with a layer of mucoadhesive polymer.

16. The formulation of claim 1 comprising tri layer tablets comprising a bioadhesive outer layer blend, inner core blend, and bioadhesive outer layer blend.

17. A method of administering a drug comprising orally administering to a patient in need thereof an oral dosage formulation comprising a mixture of a Class II drug and a bioadhesive polymer selected from the group consisting of microparticles of hydrophobic bioadhesive polymer containing drug and controlled release formulations of bioadhesive polymer containing drug.

18. The method of claim 17 wherein the drug is selected from the group consisting of anti-fungal drugs, antibiotics, steroids, hormones, and immunosuppressants.

19. The method of claim 18 wherein the drug is selected from the group consisting of itraconazole, fluoconazole, terconazole, ketoconazole, saperconazole, griseofulvin, griseoverdin, Danazole, Atovaquone, cyclosporine, digoxin, and spironolactone.

20. The method of claim 17 wherein the bioadhesive polymer is water-insoluble and is selected from the group consisting of polyanhydrides, poly(meth)acrylate, polyhydroxy acids, polyesters, and copolymers or mixtures thereof, blends comprising these polymers; and copolymers comprising the monomers of these polymers.

21. The method of claim 17 wherein the polymer comprises a mucoadhesive component selected from the group consisting of DOPA-anhydride polymer, DOPA-methacrylate polymers, DOPA-cellulosic based polymers, DOPA-acrylic acid polymers, anhydride oligomers, metal oxides, and DOPA grafted anhydrides.

22. The method of claim 17 wherein the formulation releases at least about 40% of the drug from the microparticles into a fluid of the gastrointestinal tract, or into water, in less than about 60 minutes.

23. The method of claim 17, wherein the composition is in a form selected from the group consisting of tablets, capsules, minitabs, filled tablets, osmotic devices, slurries, dispersions, and suspensions.

24. The method of claim 17 wherein the drug is in the form of particles.

25. The method of claim 17 wherein drug is incorporated into polymer at a loading selected from the group consisting of from 1 to 90% w/w, from 1 to 50 % w/w, from 20 to 70% w/w, from 40 to 60% w/w, from 30 to 40% w/w and preferably in a range from 20% to 30% w/w.

26. The method of claim 17 wherein 80% of the drug is released in 90 minutes in vitro.

27. The method of claim 17 further comprising a permeation or asorption enhancer.

28. The method of claim 17 wherein the polymer matrix is porous.

29. The method of claim 17 wherein the formulation comprises a coating selected from the group consisting of bioadhesive coatings, enteric coatings, sugar coatings, and water-soluble polymer coatings.

30. The method of claim 17 wherein the formulation comprises a tablet having a drug core and layers of mucoadhesive coating thereon.

31. The method of claim 17 wherein the formulation comprises a table comprising multiple monolithic layers, separated by slow dissolving passive matrices, coated with a moisture-protective polymer, and sealed peripherally with a layer of mucoadhesive polymer.

32. The method of claim 17 wherein the formulation comprises trilayer tablets comprising a bioadhesive outer layer blend, inner core blend, and bioadhesive outer layer blend.
